(19) United States
(12) Patent Application Publication
(10) Pub. No.: US 2024/0150413 A1
(43) Pub. Date: May 9, 2024

(54) COMPOSITIONS AND METHODS FOR DOMINANT ANTIVIRAL THERAPY

(71) Applicant: Massachusetts Institute of Technology, Cambridge, MA (US)

(72) Inventors: Alexi G. Choueiri, Cambridge, MA (US); Yong Qian, Cambridge, MA (US); Bowen Li, Cambridge, MA (US); Robert S. Langer, Cambridge, MA (US); Daniel G. Anderson, Cambridge, MA (US); Edward S. Boyden, Cambridge, MA (US)

(73) Assignee: Massachusetts Institute of Technology, Cambridge, MA (US)

(21) Appl. No.: 18/549,951

(22) PCT Filed: Mar. 11, 2022
(86) PCT No.: PCT/US2022/019896

§ 371 Date: Sep. 11, 2023

Related U.S. Application Data
(60) Provisional application No. 63/160,219, filed on Mar. 12, 2021.

(51) Int. Cl. C07K 14/005; A61K 38/16; A61K 38/46; A61P 31/14; C12N 9/22

(52) U.S. Cl. CPC C07K 14/005; A61K 38/162; A61K 38/465; A61P 31/14; C12N 9/22; C07K 2319/735; C12N 2770/20022; C12N 2770/20033

(57) ABSTRACT

In aspects, the invention provides novel compositions and methods for dominant inhibition of viral infection.

### RELATED APPLICATIONS

This application claims benefit under 35 U.S.C. § 119(e) of U.S. Provisional application Ser. No. 63/160,219 filed Mar. 12, 2021 the disclosure of which is incorporated by reference herein in its entirety.

### FIELD OF THE INVENTION

The invention relates, in part, to novel compositions and methods of suppressing a viral infection.

### BACKGROUND

Current antiviral therapies include antiviral drugs (also referred to as small molecules), small interfering RNAs (siRNAs), CRISPR, and antibodies. Because mutation is inherent in viral replication, there is a risk of a subject developing resistance to an antiviral therapy due to emergence of viral escape mutants that are resistant to the antiviral therapy being used. Mutation allows viruses to evade antiviral therapies on multiple fronts. For example, a mutation in an siRNA or CRISPR target site sequence may allow a viral mutant to escape recognition and evade destruction. A mutation may also confer resistance by altering a viral protein recognized by an antibody, or by preventing a viral protein from binding an inactivating small molecule. Once resistant viral escape mutants have arisen, there is then a risk of their being transmitted to multiple subsequent subjects.

Additional challenges associated with current antiviral therapies include complexities of target choice and sequence design including the potential need for targeting multiple sites within a viral genome; specificity; off-target effects; therapeutic dosage; safe and efficient cellular delivery and uptake; immunostimulation; pathology, epidemiology, and kinetics of infection; and obscure or incompletely understood mechanisms of infection and neutralization escape (Quershi et al., Rev Med Virol 28: e1976, 2018; Lee, Molecules 24, 2019; Salazar et al., Vaccines 2:19, 2017; Strasfeld and Chou, Infect Dis Clin North Am 24(2) 2010; Parvathaneni and Gupta, Life Sci 259, 2020).

### SUMMARY OF THE DISCLOSURE

According to an aspect of the invention, a composition for suppressing a viral infection is provided, the composition including a nucleic acid molecule encoding a fusion protein, wherein the fusion protein includes a protein capable of self-assembling/oligomerizing with a molecule of a viral particle operatively linked to a molecule capable of inhibiting a function of the viral particle. In some embodiments, the molecule of the viral particle is a viral protein. In certain embodiments, the molecule of the viral particle is a viral nucleic acid. In certain embodiments, the molecule of the viral particle is a portion of a complex including a viral protein and a viral nucleic acid. In some embodiments, the viral infection is an RNA virus infection, a DNA virus infection, a coronavirus infection, or a retroviral infection. In some embodiments, the coronavirus infection is a SARS-CoV-2 infection. In certain embodiments, the nucleic acid molecule is an mRNA molecule. In some embodiments, the function of the viral particle is reproduction of the viral particle within a cell. In some embodiments, the function of the viral particle includes one or more of assembling a new viral particle, releasing a new viral particle from a cell, and packaging a viral genome. In some embodiments, the protein capable of self-assembling/oligomerizing with a protein of the viral particle includes a protein of the viral particle. In certain embodiments, wherein the protein capable of self-assembling/oligomerizing with the viral particle protein is a nucleocapsid (N) protein. In certain embodiments, the viral nucleocapsid protein is capable of forming a complex with one or more nucleic acid molecules. In some embodiments, the fusion protein is capable of self-assembling/oligomerizing with a viral nucleocapsid protein and complexing with one or more nucleic acid molecules of the virus genome. In some embodiments, the nucleic acid molecule includes SEQ ID NO: 5. In certain embodiments, the nucleic acid molecule includes a sequence encoding one or more of SEQ ID NO: 6, SEQ ID NO: 7, SEQ ID NO: 8, SEQ ID NO: 9, SEQ ID NO: 10, SEQ ID NO: 11, SEQ ID NO: 12, and SEQ ID NO: 13. In some embodiments, the nucleic acid molecule includes a sequence encoding SEQ ID NO: 15. In some embodiments, the fusion protein includes SEQ ID NO: 4 or a functional fragment thereof. In certain embodiments, the fusion protein includes SEQ ID NO: 15. In some embodiments, the fusion protein includes one or more of SEQ ID NO: 6, SEQ ID NO: 7, SEQ ID NO: 8, SEQ ID NO: 9, SEQ ID NO: 10, SEQ ID NO: 11, SEQ ID NO: 12, and SEQ ID NO: 13. In some embodiments, the molecule capable of inhibiting a function of the viral particle is a protease, a peptide, an antibody, a lipase, or a nuclease. In some embodiments, the N protein is a coronavirus N protein, and optionally is a SARS-CoV-2 N protein. In certain embodiments, the operatively linked viral particle protein is not a capsid protein. In some embodiments, the nuclease is a calcium (Ca²⁺)-dependent nuclease. In some embodiments, in the presence of an intracellular concentration of Ca²⁺ the nuclease is incapable of an enzymatic activity and in the presence of an extracellular concentration of Ca²⁺, the nuclease is capable of the enzymatic activity. In certain embodiments, the intracellular concentration of Ca²⁺ is less than 1 micromolar (μM) and the extracellular concentration of Ca²⁺ is greater than 1 millimolar (mM). In certain embodiments, the nuclease is capable of degrading a polynucleotide component of the virus particle. In some embodiments, the nuclease is an RNase, a DNase, or a micrococcal nuclease. In some embodiments, the nuclease is Staphylococcus nuclease (SN). In some embodiments, the nuclease is RNase HI. In certain embodiments, the sequence of the nucleic acid molecule is human-codon-optimized. In certain embodiments, a means for the operative linkage is a direct linkage or includes a linker sequence. In some embodiments, the linker sequence includes a number of amino acids in one or more of the following ranges 1-20, 10-40, 20-60, 30-80, 40-100, or 50-120 amino acids. In some embodiments, the number of amino acids in the linker sequence is between 1 and 30, or 1 and 50, or 1 and 100 amino acids. In some embodiments, the protein capable of self-assembling/oligomerizing is directly linked to the molecule capable of inhibiting the function of the viral particle. In certain embodiments, the fusion protein further includes a detectable moiety. In certain embodiments, the detectable moiety includes a fluorescent moiety. In some embodiments, the detectable moiety includes a fluorescent protein. In some embodiments, the fusion protein also includes a delivery agent. In certain embodiments, the delivery agent is a nanoparticle, a polymeric nanoparticle, a dendrimer, an inorganic nanoparticle, a liposome, a lipid nanoparticle, a lipid-based nanoparticle (LNP), a lipid-polymer hybrid nanoparticle, a vector, a viral vector, a virus-like particle, a bioconjugated nanoparticle, a modified nanoparticle variant, a protein nanocage, or a DNA origami nanostructure. In some embodiments, the delivery agent is a nanoparticle including a cationic polyplex. In some embodiments, the delivery agent is a nanoparticle including a hyperbranched poly(beta amino esters) (hPBAEs). In some embodiments, the nanoparticle is capable of delivering the composition to a lung tissue. In certain embodiments, the nucleic acid molecule encoding the fusion protein is an exogenous nucleic acid molecule.

According to another aspect of the invention, a formulation that includes any embodiment or a combination of two or more embodiments of an aforementioned composition of the invention is provided. In some embodiments, the formulation is formulated for delivery by oral administration, inhalation delivery, intranasal delivery, intravenous administration, intrathecal administration, intramuscular injection, or subcutaneous injection.

According to another aspect of the invention, a cell that includes any embodiment or a combination of two or more embodiments of an aforementioned composition of the invention is provided. In some embodiments, the cell is a plant cell. In some embodiments, the cell is a vertebrate cell, optionally a mammalian cell. In certain embodiments, the cell is in a subject. In certain embodiments, the cell is one or more of a reproductive cell, a stem cell, an embryonic cell, and a transgenic cell. In some embodiments, the cell is a somatic cell. In certain embodiments, the cell is a cultured cell.

According to another aspect of the invention, a fusion protein that includes any embodiment or a combination of two or more embodiments of an aforementioned composition of the invention is provided.

According to another aspect of the invention, a cell that includes any embodiment or a combination of two or more embodiments of an aforementioned fusion protein of the invention is provided. In some embodiments, the cell is a plant cell. In some embodiments, the cell is a vertebrate cell, optionally a mammalian cell. In certain embodiments, the cell is in a subject. In some embodiments, the cell is one or more of a reproductive cell, a stem cell, an embryonic cell, and a transgenic cell. In some embodiments, the cell is a somatic cell. In certain embodiments, the cell is a cultured cell.

According to another aspect of the invention, a viral particle that includes any fusion protein or nucleic acid encoding any embodiment or a combination of two or more embodiments of an aforementioned fusion protein of the invention is provided.

According to another aspect of the invention, a method of treating a subject known to have, suspected of having, or at risk of having a viral infection is provided, the method including administering to the subject a composition including (i) a nucleic acid molecule encoding a fusion protein, wherein the fusion protein includes a protein capable of self-assembling/oligomerizing with a molecule of a viral particle operatively linked to a molecule capable of inhibiting a function of the viral particle, and (ii) a delivery molecule, in an amount effective to treat the viral infection. In certain embodiments, the administered composition includes any embodiment or a combination of two or more embodiments of an aforementioned composition of the invention. In some embodiments, the molecule of the viral particle is a viral protein. In certain embodiments, the molecule of the viral particle is a viral nucleic acid. In some embodiments, the molecule of the viral particle is a portion of a complex including a viral protein and a viral nucleic acid. In some embodiments, the delivery molecule includes a nanoparticle. In certain embodiments, the subject is a vertebrate. In certain embodiments, the vertebrate is a mammal, optionally a human. In certain embodiments, the viral infection is an RNA virus infection, a DNA virus infection, a coronavirus infection, or a retroviral infection. In some embodiments, the viral infection is a coronavirus infection, optionally a SARS-CoV-2 infection. In some embodiments, a means for the administration includes an inhalation method. In some embodiments, the inhalation method includes use of a nebulizer. In certain embodiments, a means for the administration is oral delivery, intravenous delivery, intranasal delivery, intrathecal delivery, intramuscular injection, or subcutaneous injection. In certain embodiments, the nucleic acid molecule is an mRNA molecule. In some embodiments, the nucleic acid molecule encoding the fusion protein is an exogenous nucleic acid molecule.

According to another aspect of the invention, a method of treating a subject known to have, suspected of having, or at risk of having a viral infection is provided, the method including administering to the subject a composition in an amount effective to treat the viral infection, wherein the composition includes a nucleic acid molecule encoding a fusion protein including a protein capable of self-assembling/oligomerizing with a molecule of a viral particle, wherein the protein is operatively linked to a molecule capable of inhibiting a function of the viral particle, and wherein a means of administering the composition includes a physical delivery means. In some embodiments, the molecule of the viral particle is a viral protein. In certain embodiments, the molecule of the viral particle is a viral nucleic acid. In certain embodiments, the molecule of the viral particle is a portion of a complex including a viral protein and a viral nucleic acid. In some embodiments, the subject is a vertebrate. In certain embodiments, the vertebrate is a mammal, optionally a human. In some embodiments, the viral infection is an RNA virus infection, a DNA virus infection, a coronavirus infection, or a retroviral infection. In some embodiments, the viral infection is a coronavirus infection, optionally a SARS-CoV-2 infection. In some embodiments, the administered composition includes any embodiment or a combination of two or more embodiments of an aforementioned composition of the invention. In certain embodiments, the nucleic acid molecule is an mRNA molecule. In certain embodiments, the physical delivery method includes one or more of heat shock delivery, electroporation, biolistics, microinjection, sonoporation, photoporation, magnetofection, needle injection, jet injection, electrofection, hydrofection, and optofection. In some embodiments, the administration delivers the composition into a cell of the subject, optionally into a germline cell of the subject. In some embodiments, the cell is one or more of a germline cell, an embryonic cell, a stem cell, or a reproductive cell. In certain embodiments, the subject is a plant. In some embodiments, the administered composition further includes a delivery molecule.

According to another aspect of the invention, a method of inhibiting infectivity of a mutated first virus is provided, the method including: expressing in a cell including a virus particle of the mutated first virus, a fusion protein including a protein capable of self-assembling/oligomerizing with a molecule of the first virus and operatively linked to a nuclease enzyme, wherein the mutated first virus includes the protein of the first virus and the composition inhibits infectivity of the mutated first virus. In some embodiments, the protein of the first virus is a nucleocapsid (N) protein. In some embodiments, a means for expressing the fusion protein in the cell includes delivering into the cell a composition including a nucleic acid molecule encoding the fusion protein. In certain embodiments, the composition also includes a delivery agent. In certain embodiments, the nucleic acid molecule is an mRNA molecule. In certain embodiments, the delivery agent includes a nanoparticle. In some embodiments, the polypeptide capable of self-assembling/oligomerizing with a molecule of the viral particle includes a protein of the first virus. In some embodiments, the molecule of the first virus is a protein of the first virus. In some embodiments, the molecule of the first virus includes a nucleic acid of the virus genome. In certain embodiments, the molecule of the first virus includes a protein/nucleic acid complex of the viral particle. In certain embodiments, the first virus is a coronavirus, optionally SARS-CoV-2. In certain embodiments, the N protein is a coronavirus N protein, and optionally is a SARS-CoV-2 N protein. In certain embodiments, the protein of the first virus is not a capsid protein. In some embodiments, the fusion protein including a polypeptide capable of self-assembling/oligomerizing with a protein of the first virus and operatively linked to a nuclease enzyme is encoded by the nucleic acid of any embodiment or a combination of two or more embodiments of an aforementioned composition of the invention. In some embodiments, the nucleic acid molecule encoding the fusion protein is an exogenous nucleic acid molecule.

### BRIEF DESCRIPTION OF THE DRAWINGS

FIG. 1A-1C provides schematic diagrams illustrating viral components, embodiments of the invention, and a function of an embodiment of the invention following incorporation into the viral particle. FIG. 1A shows a schematic illustrating a viral particle, in this instance a wild-type SARS-CoV-2 viral particle. Viral nucleocapsid proteins (N-proteins; round balls) bind to and package a viral genome (strand wrapped around round balls) within the lipid membrane (black circle outline), which comprises membrane proteins (ovals), envelope proteins (rectangles), and spike proteins (mushroom-shapes). FIG. 1B shows a schematic diagram of a composition of the invention, in this instance, a fusion protein (N-Nuc) comprising an N-protein (“N”), a linker sequence (between “N” and “Nuc”), and a nuclease (inactive, left panel; active, right panel). FIG. 1C shows schematic diagrams illustrating calcium-dependent nuclease cleavage of a viral genome following incorporation of N-Nuc fusion proteins into a SARS-CoV-2 viral particle. Intracellular calcium concentrations, [Ca²⁺]<1 μM, inhibit nuclease activity (left panel). However, the nuclease component of N-Nuc fusion proteins is active at the higher calcium concentrations within exocytosed viral particles ([Ca²⁺]>1 mM), and degrades the viral genome within the viral particle (right panel). N-proteins (round balls); viral genome (strand wrapped around round balls); lipid membrane (black circle outline); membrane proteins (ovals), envelope proteins (rectangles), and spike proteins (mushroom-shapes).

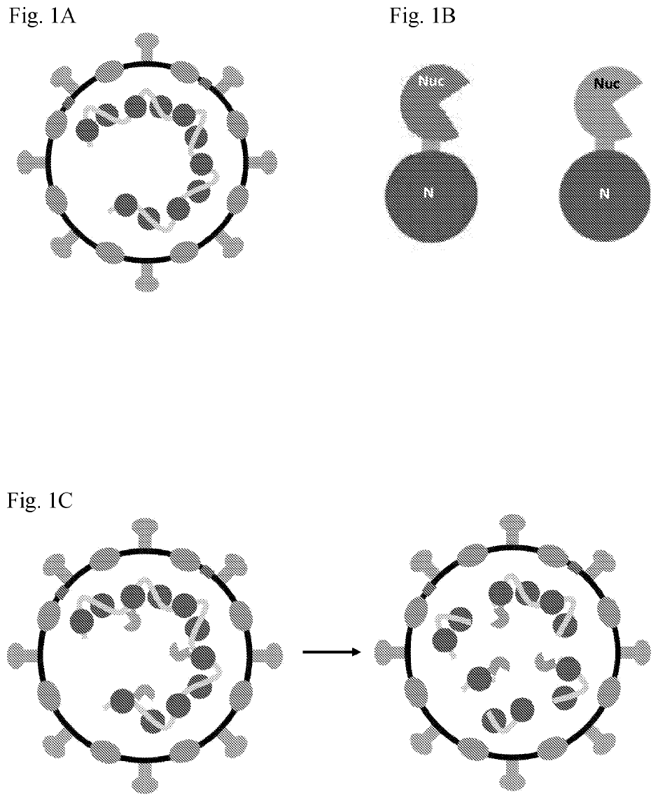

FIG. 2A-B provides a graph and photomicrographic images illustrating the baseline results of infecting cells with SARS-CoV-2. FIG. 2A shows the proportion of cells infected at high and low multiplicities of infection (MOI). FIG. 2B shows representative images of infected (left panel) and uninfected (right panel) cells stained for SARS-CoV-2 N-protein. N-protein lighter spots/material; nuclei, darker spots.

FIG. 3A-B provides a graph and photomicrographic images illustrating results of transfection experiments. FIG. 3A shows the results of transfecting cells with N-Nuc mRNA followed by SARS-CoV-2 infection. Four bars on left are High MOI; four bars on right are Low MOI. FIG. 3B shows representative images of cells transfected with either a no-mRNA control (left panel) or N-Nuc mRNA (right panel) following SARS-CoV-2 infection. N-protein lighter spots/material; nuclei, darker spots.

FIG. 4 provides photomicrographic images illustrating representative results of N-Nuc-encoding mRNA administered to SARS-CoV-2-infected cells via inhalable nanoparticles. N-protein lighter spots/material; nuclei, darker spots.

FIG. 5A-B provides photomicrographic images illustrating results of calcium-dependence nuclease activity DNA cleavage tests with wild-type and mutant fusion proteins and wild-type and mutant staphylococcal nuclease (SN) enzymes, in the presence or absence of Ca²⁺ ions. FIG. 5A shows results from calcium-dependent DNA cleavage tests with 600 ng of plasmid per reaction. FIG. 5B shows results from calcium-dependent DNA cleavage tests with 1000 ng of plasmid per reaction.

FIG. 6A-B provides a schematic diagram and bar graph illustrating results of calcium-dependence nuclease activity RNA cleavage with wild-type and mutant fusion proteins and wild-type and mutant SN enzymes, in the presence or absence of Ca²⁺ ions. FIG. 6A shows a schematic diagram of how calcium-dependent cleavage of an ssRNA reporter by a nuclease results in activation of a fluorescent reporter. FIG. 6B shows results of calcium-dependent RNA cleavage by wild-type SN enzyme (Nuc), a reduced activity SN mutant (Nuc mut), an N-protein-SN enzyme fusion protein of the invention (N-Nuc), and an N-protein-reduced activity SN mutant fusion protein (N-Nuc mut).

FIG. 7 provides photomicrographic images illustrating results of protein purification with (left panel) and without (right panel) incubation prior to purification.

FIG. 8 provides graphs illustrating results of cells transfected with N-Nuc mRNA and standard mRNA transfection reagent, rather than inhalable nanoparticles, and subsequently infected with SARS-CoV-2 at either low (0.04) or high (0.2) MOI. In each set of three bars, the left-most bar is 0 dpi; the middle bar is 1 dpi; the right-most bar is 2 dpi.

### BRIEF DESCRIPTION OF SEQUENCES

SEQ ID NO: 1-amino acid sequence of SARS-CoV-2 Nucleocapsid protein (YP_009724397.2)
MSDNGPQNQRNAPRITFGGPSDSTGSNQNGERSGARSKQRRPQGLPNNTASWFTALTQ
HGKEDLKFPRGQGVPINTNSSPDDQIGYYRRATRRIRGGDGKMKDLSPRWYFYYLGTG
PEAGLPYGANKDGIIWVATEGALNTPKDHIGTRNPANNAAIVLQLPQGTTLPKGFYAEG
SRGGSQASSRSSSRSRNSSRNSTPGSSRGTSPARMAGNGGDAALALLLLDRLNQLESKM
SGKGQQQQGQTVTKKSAAEASKKPRQKRTATKAYNVTQAFGRRGPEQTQGNFGDQEL
IRQGTDYKHWPQIAQFAPSASAFFGMSRIGMEVTPSGTWLTYTGAIKLDDKDPNFKDQV
ILLNKHIDAYKTFPPTEPKKDKKKKADETQALPQRQKKQQTVTLLPAADLDDFSKQLQQ
SMSSADSTQA.
SEQ ID NO: 2-Linker sequence
GGSGGTGSGGSG.
SEQ ID NO: 3-amino acid sequence of Staphylococcus nuclease
ATSTKKLHKEPATLIKAIDGDTVKLMYKGQPMTFRLLLVDTPETKHPKKGVEKYGPEAS
AFTKKMVENAKKIEVEFDKGQRTDKYGRGLAYIYADGKMVNEALVRQGLAKVAYVY
KPNNTHEQHLRKSEAQAKKEKLNIWSEDNADSGQ.
SEQ ID NO: 4-amino acid sequence of N-Nuc fusion protein
MSDNGPQNQRNAPRITFGGPSDSTGSNQNGERSGARSKQRRPQGLPNNTASWFTALTQ
HGKEDLKFPRGQGVPINTNSSPDDQIGYYRRATRRIRGGDGKMKDLSPRWYFYYLGTG
PEAGLPYGANKDGIIWVATEGALNTPKDHIGTRNPANNAAIVLQLPQGTTLPKGFYAEG
SRGGSQASSRSSSRSRNSSRNSTPGSSRGTSPARMAGNGGDAALALLLLDRLNQLESKM
SGKGQQQQGQTVTKKSAAEASKKPRQKRTATKAYNVTQAFGRRGPEQTQGNFGDQEL
IRQGTDYKHWPQIAQFAPSASAFFGMSRIGMEVTPSGTWLTYTGAIKLDDKDPNFKDQV
ILLNKHIDAYKTFPPTEPKKDKKKKADETQALPQRQKKQQTVILLPAADLDDFSKQLQQ
SMSSADSTQAGGSGGTGSGGSGATSTKKLHKEPATLIKAIDGDTVKLMYKGQPMTFRL
LLVDTPETKHPKKGVEKYGPEASAFTKKMVENAKKIEVEFDKGQRTDKYGRGLAYIYA
DGKMVNEALVRQGLAKVAYVYKPNNTHEQHLRKSEAQAKKEKLNIWSEDNADSGQ.
SEQ ID NO: 5-Human codon-optimized DNA sequence encoding SEQ ID NO: 4
atgtccgacaacggcccgcagaatcaacgaaacgctccccgaattacattcggtggtccatcagactccactggaagtaatcagaatgggg
aacggtcaggcgccagatcaaagcaacggcgaccgcagggtctcccgaataataccgcttcctggtttaccgccctcacccagcatggca
aagaagacctgaagtttccaaggggtcagggggtacccattaatactaattcctccccagacgaccagattggctactatcggcgagcgac
caggcgcatacggggtggtgatgggaaaatgaaggatctttcacctagatggtatttttactatctgggtacgggtccggaagcgggtctgc
cctacggcgccaacaaggacggtataatatgggttgcaactgaaggagctttgaatacacctaaggaccacattggcactcgaaatcccgc
caacaatgcagccatcgttttgcagctgccgcagggtactacacttccaaagggtttctatgccgagggatctagaggcgggtcacaggcat
cctcccggtcaagttcaagatctcgaaattcatctcgcaacagtaccccaggctcttctagaggtacctcaccggcccggatggccggcaac
ggtggcgatgctgctctcgcgctcctcctcctggaccgccttaatcagctggaaagtaagatgtccgggaagggacaacaacaacaaggc
cagacagtaactaaaaagagcgcggcagaagcgtcaaagaagccccgacaaaaaaggactgctacaaaggcatataatgtcacccagg
ctttcggccgcagaggcccagagcagacccagggcaactttggtgatcaagagttgatccggcagggcactgactataaacactggcccc
agatcgcccaattcgcaccaagtgcgagcgcatttttcgggatgtctaggattggaatggaagtcactccatctggtacctggttgacataca
caggtgcgataaagctggacgacaaggatccgaactttaaagatcaggttattttgctcaacaaacacatcgatgcttacaagactttccccc
ctactgaacccaaaaaggataaaaagaagaaagcggacgaaacacaggcgttgccgcagagacagaaaaaacagcaaactgtgactctt
cttccggcggcagacttggatgatttctctaagcaactgcagcagagtatgtcctctgctgactcaacgcaagccggcgggagtggtggtac
gggaagcgggggcagcggcgctacatcaacaaagaaactgcacaaggaaccagctactcttatcaaagctattgacggcgatactgtga
aacttatgtacaaagggcaaccaatgacatttcggcttttgctggtagatactccagaaacaaagcatcctaagaagggggtagagaaatac
ggcccggaggcgtctgcatttacaaaaaaaatggtcgaaaatgctaagaaaattgaagtcgagttcgataaaggtcagcggacggacaag
tatggaaggggattggcttatatatatgccgacggaaaaatggtaaatgaagcattggtgcgccaggggttggcaaaagtggcctacgtcta
caaaccgaataacacccatgaacagcatttgagaaaatctgaagcccaggcgaagaaagagaaactcaacatctggtcagaagataatgc
tgacagtggacaataa.
SEQ ID NO: 6-amino acid sequence of SARS-CoV-2 Nucleocapsid protein N1b domain
TASWFTALTQHGKEDLKFPRGQGVPINTNSSPDDQIGYYRRATRRIRGGDGKMKDLSPR
WYFYYLGTGPEAGLPYGANKDGIIWVATEGALNTPKDHIGTRNPANNAAIVLQLPQGTT
LPKGFYAEG.
SEQ ID NO: 7-amino acid sequence of SARS-CoV-2 Nucleocapsid protein N2b domain
TKKSAAEASKKPRQKRTATKAYNVTQAFGRRGPEQTQGNFGDQELIRQGTDYKHWPQI
AQFAPSASAFFGMSRIGMEVTPSGTWLTYTGAIKLDDKDPNFKDQVILLNKHIDAYKTF
PP.
SEQ ID NO: 8-amino acid sequence of SARS-CoV-2 Nucleocapsid protein B/N3 domain
PTEPKKDKKKKADETQALPQRQKKQQTVTLLPAADLDDFSKQLQQSMSSADSTQA.
SEQ ID NO: 9-amino acid sequence of SARS-CoV-2 Nucleocapsid protein N1a
MSDNGPQNQRNAPRITFGGPSDSTGSNQNGERSGARSKQRRPQGLPNNT.
SEQ ID NO: 10-amino acid sequence of SARS-CoV-2 Nucleocapsid protein N2a domain
GSRGGSQASSRSSSRSRNSSRNSTPGSSRGTSPARMAGNGGDAALALLLLDRLNQLESK
MSGKGQQQQGQTVT.
SEQ ID NO: 11-amino acid sequence of SARS-CoV-2 Nucleocapsid protein SB domain
PTEPKKDKKKKADETQALPQRQKKQQTV.
SEQ ID NO: 12-amino acid sequence of SARS-CoV-2 Nucleocapsid protein N3 domain
VTLLPAADLDDFSKQLQQSMSSADSTQA.
SEQ ID NO: 13-amino acid sequence of Staphylococcus nuclease minimal activity mutant
(Nuc mut)
ATSTKKLHKEPATLIKAIDGDTVKLMYKGQPMTFRLLLVDTPSTKHPKKGVEKYGPEAS
AFTKKMVENAKKIEVEFDKGQRTDKYGGGLAYIYADGKMVNEALVRQGLAKVAYVY
KPNNTHEQHLRKSEAQAKKEKLNIWSEDNADSGQ.
SEQ ID NO: 14-RNA cleavage reporter sequence
uuuuuuuu.
SEQ ID NO: 15-amino acid sequence of SARS-CoV-2 Nucleocapsid protein N2b and B/N3
domains
TKKSAAEASKKPRQKRTATKAYNVTQAFGRRGPEQTQGNFGDQELIRQGTDYKHWPQI
AQFAPSASAFFGMSRIGMEVTPSGTWLTYTGAIKLDDKDPNFKDQVILLNKHIDAYKTF
PPTEPKKDKKKKADETQALPQRQKKQQTVILLPAADLDDFSKQLQQSMSSADSTQA.

### DETAILED DESCRIPTION

An essential element of a viral infection is replicating a viral genome. As viral genomes replicate, mutations are introduced that can result in the development of mutant viral strains that may be more virulent or more infective than its originating strain, also referred to herein as its “parent strain”, or may be resistant to one or more existing therapies or vaccines. COVID-19, which emerged in late 2019, was caused by severe acute respiratory syndrome coronavirus 2 (SARS-CoV-2). With its high infectivity and mortality rates, particularly in individuals of older age and those with pre-existing health conditions, COVID-19 has rapidly expanded into a global pandemic. Within the course of the first year of the pandemic, multiple mutant strains, including but not limited to D614G, B.1.1.7, B.1.351, and P.1, arose and spread with increased rapidity compared to earlier strains of the virus.

As described herein, compositions and methods of the invention provide a dominant approach to addressing the problem of viral mutants. As used herein, the term “dominant” refers to a composition or method of treatment that acts on a virus through a mechanism that bypasses or circumvents a resistant mutation, thereby suppressing, blocking, destroying, or otherwise interfering with a viral mutant and/or propagation of a viral mutant regardless of the nature of the mutation. For example, a dominant drug can drastically suppress growth of drug-resistant viruses. A non-limiting example of dominance is through formation of mixed assemblages of proteins with highly oligomeric “sticky” properties and the incorporation of these mixed assemblages in a virus. In such circumstances, virus mutants with alterations of the N-protein may fail to assemble with an N-Nuc fusion protein of a virus, and the resulting viral particles would be highly functionally impaired. Aspects of the invention, in part, include compositions for suppressing a viral infection, the composition comprising a nucleic acid molecule encoding a fusion protein, wherein the fusion protein comprises a protein capable of self-assembling/oligomerizing with a protein of a viral particle operatively linked to a molecule capable of inhibiting a function of the viral particle.

As used herein, the term “self-assemble” refers to spontaneous association of and non-covalent interaction among at least two molecular subunits, such as a protein, to form a stable aggregate structure with a novel structure and properties (Lee and Moon, Introduction to Bionanotechnology, 2020, p. 79). As used herein, the term “oligomerize” refers to the ability of a protein to form an oligomer, a molecule comprising multiple similar or identical repeating protein units or “monomers.” Monomer units may be connected by covalent bonds or by weaker forces such as hydrogen bonds and local electric charges.

An example of a dominant approach to viral infections is capsid-targeted viral inactivation (CTVI) in which a nuclease or other effector protein is fused to a capsid protein and the fusion protein is expressed in infected cells and incorporated during virion assembly (Schumann et al., J. Virol. 2001, 75(15), 7030-7041; Zhang et al., Viruses 2016, 8, 258-270). However, CTVI has not been explored for in vivo efficacy of drug delivery and clinical application. Compositions and methods of the instant application represent an improved dominant therapy approach, including a nucleic acid encoding a fusion protein incorporating a viral N protein or functional fragment thereof and a nuclease enzyme or functional fragment thereof. Such fusion proteins may be generally referred to herein as “nucleocapsid-nuclease” or “N-Nuc” fusion proteins. The presence of the viral N protein or functional fragment thereof allows the fusion protein to be incorporated into viral particles, wherein the nuclease component of the fusion protein can suppress infectivity by degrading the viral genome within the viral particle. In some embodiments, a means of delivering the nucleic acid encoding the fusion protein is a nanoparticle comprising an mRNA encoding the fusion protein. In some embodiments, a means of delivering the nucleic acid encoding the fusion protein is a viral vector comprising a nucleic acid sequence encoding the fusion protein, or a nanoparticle comprising the viral vector. Use of the viral N protein or functional fragment thereof represents an improved dominant therapy approach because N proteins come into direct contact with the nucleic acids that make up the viral genome, thereby bringing the nuclease enzyme or functional fragment thereof and the viral genome into closer proximity. Such closer proximity may increase cleavage efficiency. Use of a nucleic acid, including but not limited to an mRNA, is compatible with art-known delivery agents, non-limiting examples of which are nanoparticles and vectors such as expression vectors. Delivery agents, as described herein, offer efficient cellular delivery and uptake, and may also provide targeted tissue and/or organ delivery.

As described herein, studies were performed demonstrating aspects of the invention in which N-Nuc fusion proteins were shown to have calcium-dependent nuclease activity against DNA and RNA in in vitro assays. Studies were also performed showing successful nanoparticle-based delivery of N-Nuc fusion protein-encoding nucleic acids, and statistically significant reductions in the proportion of infected cells at both low and high multiplicities of infection (MOI).

An additional advantage of compositions and methods of the invention versus existing strategies such as CRISPR gene-editing technologies (Freije et al. Mol. Cell 76(5) 2019, 826-837), is that compositions and methods of the invention are useful to interfere with propagation of viral mutants. Because N proteins have a role in viral genome packaging and remain with the packaged viral genome in virions, they may be efficiently incorporated into virions. As a cell safety mechanism, nucleocapsid-nuclease fusion proteins of the invention are inactive intracellularly due to nanomolar intracellular calcium (Ca²⁺) concentrations, but are active at the millimolar Ca²⁺ concentrations within a viral particle. Studies with antiviral protein-expressing transgenic mice were shown to be phenotypically normal, suggesting lack of toxicity in vivo (Schumann et al. J. Virol. 75(15) 2001, 7030-7041). In some embodiments, a fusion protein of the invention may comprise a viral protein other than an N protein. In some embodiments, a fusion protein of the invention may comprise a protein other than a nuclease. As an added advantage, nanoparticle-based delivery of mRNA is expected to be widely accessible due to large-scale mRNA synthesis capabilities, and the ability to deliver nanoparticles systemically or to target tissues through a variety of delivery methods, including but not limited to inhalation.

Some embodiments of methods of the invention comprise means referred to as N-Nuc therapy, which are capable of interfering with and inhibiting one or more different steps of a virus' life. N-Nuc therapy methods are based, in part, on the activity of N-proteins as multifunctional oligomeric proteins that have a major role in viral integration, assembly, and genome packaging [see for example Ye, Q. et al., Protein Science. 29: 1890-1901 (2020) and McBride, R., et al., Viruses. 6(8):2991-3018 (2014), the contents of which are incorporated herein by reference in their entirety]. Although not to be limited to a particular theory, N-proteins form higher order complexes that package the virus genome and targeting these proteins with N-Nuc therapy methods comprising compositions of the invention can be used to mechanistically interfere with viral assembly, integration, and packaging of the viral genome. Therefore, some embodiments of compositions and methods of the invention can be used in N-Nuc therapy methods to interfere with suppress viral infection in cells and organisms. As will be understood, in some embodiments of a composition and/or method of the invention, the N protein is in the fusion protein expressed in a cell and/or organism and may directly bind to and package the viral genome. The N protein is multifunctional and may also assemble with other viral N proteins. The N protein is a nucleoprotein that forms complex with nucleic acid (DNA/RNA) and other proteins (see Ye, Qiaozhen, et al., Protein Science. 2020; 29:1890-1901, the content of which is incorporated herein by referenced in its entirety). Thus, it will be understood that an N protein expressed in a fusion protein in a method of the invention may assemble with both viral protein and viral genetic material, e.g., the viral genome.

### Viral Infections and Symptoms

A viral infection, which may also be referred to as a viral disease, results in a cell or subject when a pathogenic virus is present in a cell or subject, or contacts a cell or subject, and infectious virus particles (virions) attach to and enter one or more cells. A viral infection in a cell, as referenced herein, means a cell into which virions have entered. A virally infected cell may be in a subject (in vivo) or obtained from a subject. In some embodiments, a virally infected cell is a cell in culture (in vitro), or is an infected cell obtained from culture. Numerous viruses are known to infect subjects and cells. Categories of infective viruses include DNA viruses and RNA viruses, including single-stranded, double-stranded, and partly double-stranded viruses. Certain types of viruses are envelope viruses, meaning they are encapsulated with a lipid membrane, which comes from an infected cell when new virus particles “bud off” from the infected cell. The lipid membrane comprises material from the infected cell's plasma membrane.

With respect to RNA viruses, positive single-stranded RNA virus families include non-enveloped viruses, such as Astroviridae, Caliciviridae and Picornaviridae; and enveloped viruses, such as Coronaviridae, Flaviviridae, Retroviridae and Togaviridae. Negative single-stranded RNA families include Arenaviridae, Bunyaviridae, Filoviridae, Orthomyxoviridae, Paramyxoviridae and Rhabdoviridae, all of which are enveloped viruses. In some embodiments of the invention, compositions and methods of the invention are applied to RNA viruses. In certain embodiments of the invention, compositions and methods of the invention are applied to an infection by a positive single-stranded RNA virus, optionally a coronaviridae infection. In some embodiments of the invention, a virus that infects a cell or subject is a SARS-CoV virus, and optionally is a SARS-CoV-2 virus. With respect to DNA viruses, double-stranded DNA virus families include non-enveloped viruses, such as Adenoviridae, Papovaviridae, and Poxviridae, and enveloped viruses such as Herpesviridae. Single-stranded DNA virus families include non-enveloped viruses, such as Parvoviridae and Anelloviridae. In some embodiments of the invention, compositions and methods of the invention are applied to DNA viruses.

As used herein, the term “viral particle” refers to an infectious viral particle or virion, whose main function is to deliver its genome (DNA or RNA) into a host cell so that its genome can be expressed, e.g., transcribed and translated, by the host cell. A complete viral particle includes one or more types of viral proteins (also referred to herein as “protein(s) of the virus”) and at least one complete copy of the viral genome (also referred to herein as a “polynucleotide component of the virus”). Several main types of viral proteins exist, including structural proteins, non-structural proteins, and regulatory and accessory proteins. Viral structural proteins include capsid proteins, envelope proteins, and membrane fusion proteins; viral non-structural proteins include proteins involved in replicon (replication complex) formation and immunomodulation (modulating the immune response of a subject to an infected cell). Viral regulatory and accessory proteins have a variety of functions, including but not limited to controlling viral gene expression in the host cell. The number and function(s) of each type of viral protein vary from virus to virus. In some embodiments, a viral protein is a nucleocapsid (N) protein, which binds to and organizes the viral genome. In some embodiments, an N protein may self-assemble or oligomerize with other N proteins. In some embodiments, the N protein may be a coronavirus N protein or a functional fragment thereof, and optionally may be a SARS-CoV-2 N protein or a functional fragment thereof. For example, though not intended to be limiting, the SARS-CoV-2 N protein or a functional fragment thereof may include one or more of the following domains: N1b (SEQ ID NO: 6), N2b (SEQ ID NO: 7), B/N3 (SEQ ID NO: 8), N1a (SEQ ID NO: 9), N2a (SEQ ID NO: 10), SB (SEQ ID NO: 11), and N3 (SEQ ID NO: 12) (Ye, et al. Protein Science 2020, doi 10.1101/2020.05.17.100685). In some embodiments of compositions of the invention, the protein capable of self-assembling/oligomerizing with a protein of the viral particle is a nucleocapsid (N) protein. In some embodiments, the protein capable of self-assembling/oligomerizing with a viral protein is not a nucleocapsid (N) protein.

It has now been shown that compositions and methods of the invention can be used to suppress viral infections. The term “suppressing a viral infection” as used herein with respect to a viral infection in a cell or subject means one or more of: treating a viral infection such that viral replication and/or propagation is statistically significantly reduced in comparison to a control treatment, and treating a viral infection such that one or more symptoms of a viral infection in a cell or subject is statistically significantly ameliorated in comparison to a control treatment. As described elsewhere herein, a control treatment may be an existing therapy known and used in the art for the viral infection being treated or for similar types of viral infections (e.g., viruses from the same family; viruses that infect similar cell or tissue types; or viral infections that result in similar symptoms), may be a placebo, or may be no treatment at all. A statistically significant reduction in viral replication in a cell or subject may be an at least 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 98%, 99%, or 100% reduction compared to a control treatment, including all percentages within that range.

A change in viral replication can be determined using a method of detecting an amount of viral particles, for example in a sample obtained from a subject, and comparing that determined amount to a control amount. In some embodiments of the invention, viral replication may be detected using molecular detection methods. Molecular detection methods are routine practices in the art and a skilled artisan would be able to use such methods in conjunction with the teachings provided herein. Non-limiting examples of molecular detection methods include PCR-based methods (e.g., endpoint PCR, quantitative PCR (qPCR), real-time PCR (rtPCR), and reverse-transcriptase PCR (RT-PCR)), CRISPR-based methods, and immunological methods (e.g., ELISA). In some aspects, suppressing a viral infection also refers to treating a viral infection such that viral replication is reduced to levels that are undetectable by molecular detection methods, though one skilled in the art will understand that suppressing a viral infection may not involve eradicating all viral particles.

The term “suppressing a viral infection” may also be used herein in reference to treating a viral infection in a cell or subject such that one or more symptoms of a viral infection in the cell or subject is statistically significantly ameliorated in comparison to the one or more symptoms in a cell or subject following a control treatment. A statistically significant amelioration of one or more symptoms of a viral infection in a cell or subject may be an at least 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 98%, 99%, or 100% reduction compared to a control treatment, including all percentages within that range. As used herein, the term “amelioration” refers to improvement in severity of one or more symptoms of a viral infection in a cell or subject compared to a control treatment or compared to the severity of one or more symptoms as determined at an earlier time point in the viral infection. Non-limiting examples of amelioration also include a reduction in number and/or severity of one or more symptoms of the viral infection in an infected cell or subject, and a reduction in overall duration of symptoms in an infected cell or subject, and a reduction in viral load in a cell or subject. Amelioration of viral infection symptoms may be evaluated and/or measured using art-known methods that will be familiar to those with ordinary skill in the art. In some aspects, suppressing a viral infection also refers to treating a viral infection such that symptoms of an infection in a subject are eliminated or apparently eliminated compared to symptoms previously exhibited by a subject, though it will be understood that in other aspects, one or more symptoms of a viral infection, which may also be referred to herein as a viral disease, may persist in a cell or subject despite the viral infection having been suppressed.

A viral infection in a subject may be symptomatic or asymptomatic. A symptomatic viral infection may result in clinical symptoms in a subject infected with the virus that may be detected and assessed using an embodiment of a method of the invention. Non-limiting examples of clinical symptoms include, but are not limited to, fever, shortness of breath, difficulty breathing, loss of sense of taste and/or smell, low blood oxygenation saturation, chills, vomiting, diarrhea, headache, muscle aches/pain, weakness, loss of appetite, malaise, nasal congestion, body aches, cough, sore throat, runny nose, and sneezing. It will be understood that presence, absence, and/or severity of one or more symptoms of a viral infection may be determined and/or assessed in an infected subject. Severity of a viral infection varies with different viruses and in different subjects. For example, a first subject with a viral infection may exhibit one or more symptoms such as, fever, chills, cough, etc. and a second subject with a more severe infection with the virus may exhibit some or all of the symptoms of the first subject, and also one or more of symptoms such as but not limited to trouble breathing, confusion, inability to stay awake, bluish lips or face, pain or pressure in chest, and significantly low blood oxygen saturation. It will be understood that clinical symptoms in a subject with a viral infection can be assessed and the symptoms identified by a health-care professional.

### Inhibiting Infectivity of Viruses and Mutant Viruses

An essential property of a virus is infectivity, which requires intact and functional viral proteins and an intact, functional genome that can be replicated by a host cell. As used herein, “infectivity” refers to the ability of a first virus to successfully infect a host cell, which involves successful completion of multiple steps. For example, though not intended to be limiting, to infect a host cell, a viral particle must interact with and/or bind to a receptor or other cell-surface protein of the host cell and be successfully internalized by the host cell. The viral envelope or capsid must disassemble and release the viral genome and any other viral proteins, and must successfully “hijack” the nucleic acid replication and translation machinery of the host cell in order to reproduce multiple copies of the viral genome and viral proteins. Various viral proteins must package those new viral genomes into newly assembling viral particles within the host cell, and the newly assembled viral particles (a second virus) must be released from the host cell. N proteins are involved in packaging viral genomes into viral particles and accompany the new viral genomes within the newly-assembled viral particles.

Inhibiting infectivity, as used herein refers to interfering with or blocking one or more aspects of the viral property of infectivity. It has now been determined that compositions and methods of the invention can be used to inhibit infectivity of a virus. A molecule capable of inhibiting a function of the viral particle is a molecule that can interfere with or block infectivity by interacting with an element of the viral particle. Non-limiting examples of means of blocking infectivity include disrupting a viral envelope (e.g., by a lipase breaking down membrane lipids); preventing a viral particle from interacting with cell-surface proteins (e.g., by an antibody binding to a viral capsid or envelope protein); degrading a viral protein (e.g., by a protease); and degrading a viral genome (e.g., by a nuclease cleaving the viral genome RNA or DNA into smaller oligonucleotides that cannot be transcribed or replicated in a host cell).

In certain embodiments of compositions and methods of the invention, a molecule capable of inhibiting a function of the viral particle is a protease, a peptide, an antibody, a lipase, or a nuclease. A protease is an enzyme that catalyzes breakdown of proteins and polypeptides into smaller polypeptides or single amino acids. A peptide may be a full-length protein and may also be a functional fragment of a full-length protein, and/or a functional variant thereof. An antibody is a protein capable of recognizing and binding to a specific protein or protein fragment (antigen). A lipase is an enzyme that catalyzes breakdown of lipids into free fatty acids and glycerol. A nuclease is an enzyme that degrades RNA or DNA or both by catalyzing cleavage of the RNA or DNA backbone.

It is known in the art that viruses may mutate during replication in a host cell, and that mutant variants of a first virus can result in resistance to anti-viral therapies such as antibodies and small molecules. It has now been determined that compositions and methods of the invention can be used to inhibit production and/or spread of viruses that are mutated variants of a first virus. As used herein the term “parent virus” may be used to indicate a first virus from which a second virus, also referred to herein as a mutated first virus, arises. Aspects of the invention, in part, include methods of inhibiting infectivity of a mutated first virus. Methods of the invention may comprise expressing in a cell comprising a virus particle of the mutated first virus, a fusion protein comprising a polypeptide capable of self-assembling/oligomerizing with a viral protein of the first virus and operatively linked to a nuclease enzyme, wherein the mutated first virus comprises the viral protein of the first virus and the composition inhibits infectivity of the mutated first virus. In some embodiments of methods of the invention, the viral protein is a nucleocapsid (N) protein. In some embodiments, the N protein is a coronavirus N protein, and optionally is a SARS-CoV N protein or a SARS-CoV-2 N protein. In some embodiments, the protein of the first virus is not a capsid protein. In some embodiments, the fusion protein comprising a polypeptide capable of self-assembling/oligomerizing with a dominant protein of the first virus and operatively linked to a nuclease enzyme, is encoded by a nucleic acid molecule as described elsewhere herein. For example, in some embodiments, a first virus has been produced by a first host cell expressing a composition of the invention, and the genome of the first virus has been packaged with N-Nuc fusion proteins encoded by a nucleic acid molecule of the invention capable of degrading the genome within the capsid, thereby rendering the first virus non-infective if internalized by a second host cell. Notably, a fusion protein of the invention is capable of degrading the viral genome and rendering it non-infective even if it contained mutations relative to the parental viral genome from which it was copied.

### Nucleic Acids

As used herein, the term “nucleic acid”, “nucleic acid molecule”, or “polynucleotide” refers to a polymer comprising multiple nucleotide monomers. The term “nucleotide” as used herein includes a phosphoric ester of nucleoside—the basic structural unit of nucleic acids (DNA or RNA). A nucleic acid may be either single stranded, or double stranded with each strand having a 5′ end and a 3′ end. A nucleic acid may be RNA (including but not limited to mRNA or genomic RNA of an RNA virus), DNA (including but not limited to cDNA, genomic DNA, or genomic DNA of a DNA virus), or hybrid polymers (e.g., DNA/RNA). The terms “nucleic acid” and “nucleic acid molecule” do not refer to any particular length of polymer. Nucleic acid molecules of the invention may be at least 100, 200, 300, 400, 500, 600, 700, 800, 900, 1000, 2000, 5000, or 10,000 nucleotides in length. The term “sequence,” used herein in reference to a nucleic acid molecule, refers to a contiguous series of nucleotides that are joined by covalent bonds, such as phosphodiester bonds. A nucleic acid molecule may be chemically or biochemically synthesized, or may be produced by or isolated from a subject, cell, tissue, or other biological sample or source that comprises, or is believed to comprise, nucleic acid sequences including, but not limited to RNA, mRNA, and DNA. Further, this disclosure contemplates that a nucleic acid molecule of the invention may comprise at least one modified nucleotide, which may be incorporated into a polynucleotide by, for example, chemical synthesis. Such modified nucleotides may confer additional desirable properties absent or lacking in the natural nucleotides, and polynucleotides comprising modified nucleotides may be used in the compositions and methods of the invention.

Some embodiments of compositions and methods of the invention include a nucleic acid molecule comprising a sequence encoding a viral protein or a functional fragment thereof. For example, though not intended to be limiting, the nucleic acid molecule sequence may comprise a sequence encoding SEQ ID NO: 1, or may comprise a sequence encoding at least one or more of SEQ ID NOs: 6-13 and 15. In some aspects, the nucleic acid molecule of the invention comprises a sequence encoding a fusion protein, wherein the fusion protein comprises a protein or functional fragment thereof capable of self-assembling/oligomerizing with a viral protein operatively linked to a molecule or functional fragment thereof capable of inhibiting a function of the viral particle. In some embodiments, a nucleic acid molecule encoding the fusion protein encodes SEQ ID NO: 4. In other embodiments, the nucleic acid molecule encoding the fusion protein comprises SEQ ID NO: 5. In some embodiments, the nucleic acid molecule encodes at least one or more of SEQ ID NOs: 1-3, 6-13, and 15. In other embodiments, the nucleic acid molecule encodes at least one or more of SEQ ID NOs: 2, 3, 6-13, and 15. In some embodiments, the nucleic acid molecule encodes at least one or more of SEQ ID NOs: 3, 6-13, and 15. Certain non-limiting examples of amino acid sequences encoded by the nucleic acid molecule include fusion protein sequences comprising: SEQ ID NOs: 2, 3, and 6; SEQ ID NOs: 2, 3, and 7; SEQ ID NOs: 2, 3, and 8; SEQ ID NOs: 2, 3, and 9; SEQ ID NOs: 2, 3, and 10; SEQ ID NOs: 2, 3, and 11; SEQ ID NOs: 2, 3, and 12; SEQ ID NOs: 2, 3, and 15; SEQ ID NOs: 3 and 6; SEQ ID NOs: 3 and 7; SEQ ID NOs: 3 and 8; SEQ ID NOs: 3 and 9; SEQ ID NOs: 3 and 10; SEQ ID NOs: 3 and 11; SEQ ID NOs: 3 and 12; SEQ ID NOs: 3 and 15; SEQ ID NOs: 3, 6, and 9; SEQ ID NOs: 3, 7, and 10; SEQ ID NOs: 3, 7, and 8; SEQ ID NOs: 3, 10, and 15; SEQ ID NOs: 1, 2, and 13; SEQ ID NOs: 2 and 13; and SEQ ID NOs: 13 and 15. In some aspects of the invention, the nucleic acid molecule is an mRNA molecule.

It is known in the art that different organisms exhibit bias towards use of certain codons over others for the same amino acid. Therefore, in some embodiments of the invention, sequences of nucleic acid molecules of the invention are codon-optimized, meaning that the codons of the nucleic acid sequence are tailored for the codon preferences of the organism in which the nucleic acid molecule will be expressed. In some embodiments, sequences of nucleic acid molecules of the invention are human-codon-optimized, i.e., optimized for expression in human cells.

### Proteins and Fusion Proteins

Aspects of the invention include compositions encoding and methods using full-length proteins or functional fragments thereof. The terms “protein” and “polypeptide” are used interchangeably herein and thus the term polypeptide may be used to refer to a full-length protein and may also be used to refer to a fragment of a full-length protein. A protein is a polymer of amino acids, and as used herein refers to at least two amino acids. “Functional fragments” as referred to herein are fragments of a full-length protein that retain at least a portion of a distinct functional capability of the polypeptide. A portion is at least 5%, 10%, 15%, 20%, 25%, 30%, 35%, 40%, 45%, 50%, 55%, 60%, 65%, 70%, 75%, 80%, 85%, 90%, 95%, or 100% of the functional capability (including all integer values in between). Functional capabilities that can be retained in a fragment include interaction with nucleic acids, and interaction with other polypeptides or fragments thereof. In some embodiments, functional fragments of a polypeptide may be encoded by a nucleic acid molecule of the invention, or may be synthesized using art-known methods, and tested for function using the methods exemplified herein. Full-length proteins and functional fragments that are useful in methods and compositions of the invention may be recombinant polypeptides.

A fragment of a full-length polypeptide may comprise at least up to n−1 contiguous amino acids of the full-length polypeptide having a consecutive sequence found in a wild-type polypeptide or in a modified polypeptide sequence as described herein (with “n” equal to the number of amino acids in the full-length polypeptide). Thus, for example, a fragment of a 150 amino acid-long polypeptide would be at least 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, or 149 (including each integer in between) contiguous amino acids of the 150 amino acid polypeptide. In some embodiments, a fragment includes the C-terminal region of a polypeptide; in some embodiments, a fragment includes the N-terminal region of the polypeptide.

The term “variant” as used herein in the context of polypeptide molecules and/or polynucleotide molecules, describes a molecule with one or more of the following characteristics: (1) the variant differs in sequence from the molecule of which it is a variant (also referred to herein as a “parent molecule”), (2) the variant is a fragment of the molecule of which it is a variant and is identical in sequence to the fragment of which it is a variant, and/or (3) the variant is a fragment and differs in sequence from the fragment of the molecule of which it is a variant. As used herein, the term “parent” in reference to a sequence means a sequence from which a variant originates.

A “modified” wild-type or mutant full-length polypeptide or polypeptide that is a fragment thereof may include deletions, point mutations, truncations, amino acid substitutions and/or additions of amino acids or non-amino acid moieties. Modifications of a polypeptide may be made by modification of the nucleic acid molecule of the invention that encodes the polypeptide or alternatively, modifications may be made directly to the polypeptide, such as by cleavage, addition of a linker molecule, addition of a detectable moiety, such as a fluorescent label, and the like. Modifications also embrace fusion proteins comprising all or part of the polypeptide's amino acid sequence. Modifications to polypeptides may also include one or more post-translational modifications to amino acids, including but not limited to phosphorylation, acetylation, O-/N-linked glycosylation, amidation, hydroxylation, methylation, ubiquitination, sulfation, and addition of pyroglutamic acid. Means of adding and identifying post-translational modifications are known and routinely practiced in the art, see for example, Virag, et al. Chromatographia doi/10.1007/s10337-019-03796-9, 201).

As used herein the term “modified” or “modification” in reference to a polypeptide sequence or a polynucleotide sequence refers to a change of 1, 2, 3, 4, 5, 6, 7, 8, 9, 10, 11, 12, 13, 14, 15, 16, 17, 18, 18, 20, 21, 22, 23, 24, 25, or more amino acids or nucleic acids, respectively in the sequence as compared to the parent polypeptide, or encoding nucleic acid sequence. As used herein, a sequence change or modification may be one or more of a substitution, deletion, insertion or a combination thereof. For example, though not intended to be limiting: the amino acid sequence of a functional variant N polypeptide may be identical to the sequence set forth as SEQ ID NO: 1 except that it has one, two, three, four, five, or more amino acid substitutions, deletions, insertions, or combinations thereof.

In general, modified polypeptides (e.g. modified wild-type or mutant polypeptides) may include polypeptides that are modified specifically to alter a feature of the polypeptide unrelated to its physiological activity. For example, cysteine residues can be substituted or deleted to prevent unwanted disulfide linkages. A residue may be added at the N or C-terminal end of the polypeptide, for example, a cysteine (C) or other amino acid residue may be added at the extreme C-terminal end of a polypeptide. Modifications can be made in a nucleic acid molecule of the invention by selecting and introducing an amino acid substitution, deletion, or addition. Modified polypeptides then can be tested for one or more activities (e.g., nuclease activity, etc.) to determine which modification provides a modified polypeptide with the desired properties.

The skilled artisan will also realize that conservative amino acid substitutions may be made in a polypeptide to provide functionally equivalent polypeptides, i.e., a modified N protein or SN nuclease that retains a functional capability of an un-modified N protein or SN nuclease, respectively, in a composition or method of the invention. As used herein, a “conservative amino acid substitution” refers to an amino acid substitution that does not alter the relative charge or size characteristics of the polypeptide in which the amino acid substitution is made. Modified polypeptides can be prepared according to methods for altering polypeptide sequence and known to one of ordinary skill in the art such. Exemplary functionally equivalent polypeptides include conservative amino acid substitutions of an N protein or SN nuclease, or respective fragments thereof, such as a modified N protein or SN nuclease. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D. One of ordinary skill in the art will know how to use 1, 2, 3, 4, 5 or more conservative amino acid substitutions in conjunction with methods and compositions of the invention.

Conservative amino-acid substitutions typically are made by alteration of a nucleic acid encoding the polypeptide. Such substitutions can be made by a variety of methods known to one of ordinary skill in the art. For example, amino acid substitutions may be made by PCR-directed mutation, site-directed mutagenesis, or by chemical synthesis of a nucleic acid molecule encoding the polypeptide, e.g., an mRNA. Where amino acid substitutions are made to a small fragment of a polypeptide, the substitutions can be made by directly synthesizing the polypeptide. The activity of functionally equivalent fragments of polypeptides can be tested by synthesizing an mRNA encoding the altered polypeptide and expressing the mRNA in an appropriate host cell, or by cloning a gene encoding the altered polypeptide into a bacterial or mammalian expression vector, introducing the vector into an appropriate host cell, expressing the altered polypeptide, and testing for a functional capability of the polypeptide as disclosed herein.

In aspects, compositions and methods of the invention include a fusion protein encoded by a nucleic acid molecule of the invention. As used herein, “fusion protein” refers to a non-naturally occurring protein comprising amino acid sequences from at least two different proteins. In some embodiments, the fusion protein includes a protein or functional fragment thereof capable of self-assembling and/or oligomerizing with a viral protein operatively linked to a molecule capable of inhibiting a function of the viral particle. In embodiments, the protein capable of self-assembling/oligomerizing with a protein of the viral particle is a nucleocapsid (N) protein. In embodiments of the invention, the fusion protein comprises SEQ ID NO: 4 or a functional fragment thereof. In some embodiments, the fusion protein comprises at least one of SEQ ID NOs: 6-13 and 15. In other embodiments, the fusion protein comprises at least one or more of SEQ ID NOs: 1-3, 6-13, and 15. In some embodiments, the fusion protein comprises at least one or more of SEQ ID NOs: 2, 3, 6-13 and 15. In some embodiments, the fusion protein comprises at least one or more of SEQ ID NOs: 3, 6-13, and 15. Non-limiting examples of combinations of sequences that may be included in a fusion protein in a compositions and/or method of the invention are SEQ ID NOs: 2, 3, and 6; SEQ ID NOs: 2, 3, and 7; SEQ ID NOs: 2, 3, and 8; SEQ ID NOs: 2, 3, and 9; SEQ ID NOs: 2, 3, and 10; SEQ ID NOs: 2, 3, and 11; SEQ ID NOs: 2, 3, and 12; SEQ ID NOs: 2, 3, and 15; SEQ ID NOs: 3 and 6; SEQ ID NOs: 3 and 7; SEQ ID NOs: 3 and 8; SEQ ID NOs: 3 and 9; SEQ ID NOs: 3 and 10; SEQ ID NOs: 3 and 11; SEQ ID NOs: 3 and 12; SEQ ID NOs: 3 and 15; SEQ ID NOs: 3, 6, and 9; SEQ ID NOs: 3, 7, and 10; SEQ ID NOs: 3, 7, and 8; SEQ ID NOs: 3, 10, and 15; SEQ ID NOs: 1, 2, and 13; SEQ ID NOs: 2 and 13; and SEQ ID NOs: 13 and 15. In some embodiments, the operatively linked protein of the viral particle is not a capsid protein. In some embodiments, a means for the operative linkage may be a direct linkage or may include a linker sequence. In some embodiments of the invention, a linker sequence includes at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 100, or 150 amino acids, including every integer within the range. In some embodiments of the invention the length of a linker is in a range of 1-10, 1-20, 5-20, 5-50, 10-20, 10-50, 20-50, 20-100, 30-80, 40-100, or 50-120 amino acids, including every integer within the ranges.

In some embodiments, the protein capable of self-assembling/oligomerizing is directly linked to the molecule capable of inhibiting the function of the viral particle. In some embodiments, the sequence of the nucleic acid encoding a fusion protein is codon-optimized for an organism to which it will be delivered. In some embodiments, the nucleic acid sequence encoding the fusion protein is human-codon-optimized.

### Nucleases

In aspects of the invention, the molecule capable of inhibiting a function of the viral particle is a nuclease. A nuclease is an enzyme that degrades an RNA polynucleotide (an RNase) or a DNA polynucleotide (a DNase) or both by catalyzing cleavage of the RNA or DNA sugar-phosphate backbone. Therefore, in some embodiments, the nuclease is capable of degrading a polynucleotide component of the virus, including but not limited to genomic DNA or RNA, single-stranded DNA or RNA (+ or − strands), double-stranded DNA or RNA, subgenomic RNA, and mRNA. Types of nucleases include exonucleases (cleave nucleotides one at a time from the end of a polynucleotide), endonucleases (cleave within polynucleotides rather than from the ends), and endo-exonucleases that can perform both functions. Most nucleases, though not all, require a divalent cation as a cofactor, usually Mg′ (magnesium-dependent nuclease) or Ca²⁺ (calcium-dependent nuclease).

In some embodiments, the nuclease is a calcium (Ca²⁺)-dependent nuclease, and may have Ca²⁺ concentrations within which it is inactive, that is, incapable of enzymatic activity; Ca²⁺ concentrations within which it is partially active, that is, capable of some enzymatic activity; and Ca²⁺ concentrations within which it is active, that is, capable of full enzymatic activity. As is known in the art, regardless of whether cells are in vitro or in vivo, intracellular and extracellular Ca²⁺ concentrations are typically very different. In some embodiments, the intracellular concentration of Ca²⁺ is less than 1 micromolar (μM) and the extracellular concentration of Ca²⁺ is greater than 1 millimolar (mM). For example, though not intended to be limiting, in the presence of an intracellular concentration of Ca²⁺ the nuclease may be incapable of an enzymatic activity, whereas in the presence of an extracellular concentration of Ca²⁺ the nuclease may be capable of the enzymatic activity. One of skill in the art will understand that calcium-dependent nuclease cleavage provides a host safety mechanism that keeps the nuclease inactive within host cells.

In some embodiments, the calcium-dependent nuclease is a micrococcal nuclease. As used herein, a micrococcal nuclease is an endo-exonuclease that preferentially digests single-stranded DNA or RNA, especially at AT- or AU-rich regions and also digests double-stranded DNA or RNA. Micrococcal nuclease is also known in the art, and may be referred as MNase, spleen endonuclease, thermonuclease, nuclease T, micrococcal endonuclease, nuclease T′, staphylococcal nuclease (SN), spleen phosphodiesterase, Staphylococcus aureus nuclease, Staphylococcus aureus nuclease B, ribonucleate (deoxynucleate) 3′-nucleotidohydrolase). Micrococcal nuclease is an endo-exonuclease that preferentially digests nucleic acids that are single stranded. The rate of micrococcal nuclease cleavage of a single-stranded nucleic acid molecule is much higher at the 5′ side of A or T than it is at the 5′ side of G or C, so the resulting mononucleotides and oligonucleotides that are produced by micrococcal cleavage have terminal 3′-phosphates. Micrococcal nuclease can also cleave double-stranded DNA and RNA, which results in all sequences being ultimately cleaved.

In other embodiments, the nuclease is a ribonuclease (RNase), a deoxyribonuclease (DNase), or a micrococcal nuclease. Non-limiting examples of RNAses include RNase A, RNase H, RNase HI, RNase L, RNase PhyM, RNase T1, RNase T2, RNase U2, RNase V, and RNase R. Non-limiting examples of DNases include DNase I and DNase II.

### Labelling

In aspects of the invention, a fusion protein encoded by a nucleic acid molecule of the invention also comprises a detectable label, thereby enabling visual identification and localization of the fusion protein or proteins. As used herein, the term “detectable label” means a label that is encoded by the nucleic acid molecule of the invention as a region of the fusion protein or is chemically bound (e.g., covalently, or via hydrogen or ionic bonding) to the fusion protein, and can be detected through microscopy or other means of detection. In some embodiments, the detectable label comprises a fluorescent label, a luminescent label, a radiolabel, an enzymatic label, a contrast agent, a heavy metal, or a heavy element such as bromine or iodine, or metals such as gold, osmium, rhenium, etc. In some embodiments, the detectable label is a fluorescent protein or other light-emitting protein. Non-limiting examples of fluorescent proteins include: luciferase, green fluorescent protein (GFP); enhanced green fluorescent protein (EGFP), red fluorescent protein (RFP); yellow fluorescent protein (YFP), dtTomato, mCherry, DsRed, mRuby, cyan fluorescent protein (CFP); far red fluorescent proteins, etc. Numerous fluorescent proteins and their encoding nucleic acid sequences are known in the art and routine methods can be used to include such sequences in compositions and methods of the invention. The fusion protein may furthermore include more than one label. For example, each label may have a particular or distinguishable fluorescent property, e.g., distinguishable excitation and emission wave-lengths.

### Treatment

The invention, in some aspects, relates to compositions for and methods of treating a subject known to have, suspected of having, or at risk of having a viral infection. Some embodiments of methods of the invention include administering to the subject a composition comprising: an mRNA encoding a fusion protein, wherein the fusion protein comprises a protein capable of self-assembling/oligomerizing with a protein or polynucleotide of a viral particle, and wherein the protein is operatively linked to a molecule capable of inhibiting a function of the viral particle, and a delivery molecule, in an amount effective to treat the viral infection. As used herein, the terms “treat”, “treated”, or “treating” when used with respect to a viral infection may refer to a prophylactic treatment that decreases the likelihood of a subject developing the infection or symptoms thereof, and also may refer to a treatment after the subject has developed the infection or symptoms thereof in order to eliminate or reduce the level of the infection or eliminate or ameliorate symptoms thereof, prevent the infection or symptoms thereof from becoming more advanced (e.g., more severe), and/or slow the progression of the infection or symptoms thereof compared to the absence of the therapy.

### Delivery

Compositions of the invention may be delivered to a cell or a subject in a formulation. As used herein, a formulation refers to a pharmaceutical preparation or pharmaceutical composition comprising a composition of the invention as well as other active or inert components that is stable and safe for administration to a subject. A formulation may also include a means for administering the pharmaceutical preparation or composition to a cell or subject.

In some embodiments, a formulation may comprise a delivery agent, a non-limiting example of which is a nanoparticle as described elsewhere herein. In some embodiments, the formulation may be an inhalation-delivery formulation, and may further comprise nanoparticles. A non-limiting example of a means for administering the inhalation-delivery formulation is a nebulizer, a machine that turns liquid into mist. In other embodiments, the formulation may be formulated for administration by oral, intravenous, intrathecal, intracavity, intrathecal, intrasynovial, buccal, sublingual, intranasal, transdermal, intravitreal, intramuscular injection, or subcutaneous injection means. Various administration means will be known to one of ordinary skill in the art that can be used to effectively deliver a formulation to increase the level of a composition of the invention in a desired cell, tissue or body region of a subject. The invention is not limited by the particular modes of administration disclosed herein. Standard references in the art (e.g., Remington's Pharmaceutical Sciences, 23rd edition, 2020) provide means of administration and formulations for delivery of various pharmaceutical preparations and formulations in pharmaceutical carriers. Other protocols which are useful for the administration of a composition of the invention will be known to one of ordinary skill in the art, in which the dose amount, schedule of administration, sites of administration, mode of administration and the like vary from those presented herein.

Formulations that can be used to deliver compositions of the invention may be administered alone, in combination with each other, and/or in combination with other drug therapies, or other anti-viral treatment regimens that are administered to subjects. A formulation used in one of the foregoing methods preferably contains an effective amount of a composition of the invention that will increase the level of a fusion protein encoded by a nucleic acid molecule of the invention to a level that produces the desired response in a unit of weight or volume suitable for administration to a subject.

It will be understood that treatment methods of the invention can be used in combination with other therapeutics. A non-limiting example of a treatment that may be selected for inclusion in an anti-viral therapeutic regimen is an antibody therapy, such as but not limited to a monoclonal antibody therapy. Non-limiting examples of antibody therapy that may be selected include administration of Bamlanivimab (LY-CoV555), casirivimab, imdevimab, a casirivimab-imdevimab combination, and convalescent plasma therapy. Another non-limiting example of a treatment that may be selected for inclusion in a therapeutic regimen is an anti-viral therapy, a non-limiting example of which comprises Veklury (remdesivir) administration; bed rest; respiratory therapy, non-limiting examples of which are supplemental oxygen administration, mechanical respiration assistance, and attachment to a respirator; acetaminophen administration; Ibuprofen administration, NSAID administration; hydration therapy; corticosteroid administration, non-limiting examples of which are dexamethasone administration, prednisone administration, and methylprednisolone administration; chloroquine administration, a non-limiting example of which comprises hydroxychloroquine administration; antibiotic administration, a non-limiting example of which comprises Azithromycin administration; vitamin D administration; anti-inflammatory administration, a non-limiting example of which comprises Olumiant (baricitinib) administration; CD24Fc recombinant fusion protein administration; synthetic antibody administration, a non-limiting example of which comprises AZD7442 (combination of two monoclonal antibodies) administration; VIR-7831(GSK4182136) administration; a respiratory-support therapy, a physical isolation therapy; a physical positioning therapy, a sedation therapy, a surgical therapy, a hydration therapy, and a physical therapy. In some embodiments, the respiratory-support therapy comprises administering oxygen to a subject, optionally high-flow oxygen administration. In certain embodiments, the respiratory-support therapy comprises one or more of intubation and ventilation of the subject.

It will be understood that in some embodiments, administration is done by a health-care professional and in certain embodiments, administration is self-administration by a subject or administration by a non-health-care individual. It will be understood that a treatment regimen may include one or more administrations of a selected treatment and more than one type of treatment may be selected for inclusion in a treatment regimen for a subject.

### Effective Amounts and Dosing

An effective amount of an mRNA or fusion protein of the invention is an amount that increases the level of the mRNA or fusion protein of the invention in a cell, tissue, or subject to a level that is beneficial for the subject. An effective amount may also be determined by assessing physiological effects of administration on a cell or subject, such as a decrease in symptoms following administration. Other assays will be known to one of ordinary skill in the art and can be employed for measuring the level of the response to a treatment. The amount of a treatment may be varied for example by increasing or decreasing the amount of the mRNA or fusion protein administered, by changing the therapeutic composition in which the mRNA or fusion protein is administered, by changing the route of administration, by changing the dosage timing, and so on. The effective amount will vary with the particular viral infection being treated, the age and physical condition of the subject being treated, the severity of the infection, the severity and duration of symptoms exhibited by the subject being treated, the duration of the treatment, the nature of the concurrent therapy (if any), the specific route of administration, and the like factors within the knowledge and expertise of the health practitioner. For example, an effective amount may depend upon the location and number of cells in the subject in which the mRNA or fusion protein of the invention is to be expressed. An effective amount may also depend on the location of the tissue to be treated.

Effective amounts will also depend, of course, on the particular viral infection being treated, the severity of the infection, the severity and duration of symptoms exhibited by the subject being treated, individual subject parameters including age, physical condition, size and weight, the duration of the treatment, the nature of concurrent therapy (if any), the specific route of administration and like factors within the knowledge and expertise of the health care professional. These factors are well known to those of ordinary skill in the art and can be addressed with no more than routine experimentation. It is generally preferred that a maximum dose of a composition to increase the level of an mRNA or fusion protein of the invention in a subject (alone or in combination with other therapeutic agents) be used, that is, the highest safe dose or amount according to sound medical judgment. It will be understood by those of ordinary skill in the art, however, that a subject may insist upon a lower dose or tolerable dose for medical reasons, psychological reasons or for virtually any other reasons. The manner and dosage administered may be adjusted by the individual health care professional or veterinarian, particularly in the event of any complication. The absolute amount administered will depend upon a variety of factors, including the means selected for administration, whether the administration is in single or multiple doses, and individual subject parameters including age, physical condition, size, weight, and the stage of the infection. These factors are well known to those of ordinary skill in the art and can be addressed with no more than routine experimentation.

The dose of a formulation that is administered to a subject to increase the level of an mRNA or fusion protein of the invention in cells of the subject may be chosen in accordance with different parameters, in particular in accordance with the mode of administration used and the state of the subject. Other factors include the desired period of treatment. Dosing may be determined with routine methods, including but not limited to clinical trials. In the event that a response in a subject is insufficient at the initial doses applied, higher doses (or effectively higher doses by a different, more localized delivery route) may be employed to the extent that the subject may tolerate.

### Delivery Agents

In aspects, compositions and methods of the invention includes a delivery agent, which as used herein, refers to a chemical, molecule, or compound capable of interacting with and/or encapsulating a nucleic acid molecule or fusion protein of the invention and facilitating entry of the nucleic acid molecule or fusion protein into a cell. Non-limiting examples of delivery agents that may be used in compositions and methods of the invention are liposomes, nanoparticles, and vectors.

In some embodiments of compositions and methods of the invention, a delivery agent may be a nanoparticle, a polymeric nanoparticle, a liposome, a lipid nanoparticle, a lipid-based nanoparticle (LNP), a lipid-polymer hybrid nanoparticle, an inorganic nanoparticle, a virus-like particle, a bioconjugated nanoparticle, a modified nanoparticle variant, a protein nanocage, or a DNA origami nanostructure. A nanoparticle is a particle of matter with an overall dimension of under 100 nanometers (nm). Nanoparticles may exhibit unique physical and chemical properties due to their small size, and may be used as vehicles for delivering nucleic acids, peptides, chemicals, or small molecules to cells or tissues or within a biological system. In some embodiments, the nanoparticle is capable of delivering a composition of the invention to a lung or lung tissue. Nanoparticles may be comprised of smaller individual organic molecular components such as polymers and lipids, may be a single inorganic particle or nanocrystal, may be a single organic nanocrystal, or may be a bioconjugated nanoparticle. Lipid-based nanoparticles include liposomes comprising phospholipids that can form unilamellar and multilamellar vesicular structures, and lipid nanoparticles (LNPs) that are liposome-like structures typically comprising cationic or ionizable lipids, phospholipids, cholesterol, and PEGylated lipids. LNPs and lipid-based nanoparticles may be further modified by the addition of functional groups, peptides, polymers, or inorganic materials. Polymeric nanoparticles may be synthesized from either natural or synthetic materials, and a wide variety of types of polymers may be used, including dendrimers, which are hyperbranched, radially symmetric polymers with a complex three-dimensional architecture that can be controlled and active functional groups that can enable conjugation of other molecules to the surface of the nanoparticle (Mitchell et al. Nat. Rev. Drug Disc. 2020). In some embodiments, a delivery agent is a nanoparticle comprising a cationic polyplex or a hyperbranched poly(beta amino esters) (hPBAEs) (Patel et al. Adv. Mater. 2019, 1805116). Lipid-polymer hybrid nanoparticles may be assembled that comprise both lipids and polymers. Inorganic nanoparticles may include materials such as silica, gold, titanium, iron oxides, or quantum dots (inorganic semiconductor nanocrystals). Organic nanocrystals are crystals of nanometer size of an organic compound. Bioconjugated nanoparticles are conjugates of an inorganic particle and one or more biological molecules, such as a bovine serum albumen (BSA)-conjugated gold nanoparticle. Virus-like particles, protein particles derived from a viral capsid, may also be used as nanoparticles. Protein nanocages are formed by self-assembly of protein subunits and may be used to deliver molecular cargos to cells (Bhaskar and Lim, NPG Asia Materials 9, e371, 2017). DNA origami nanostructures are self-assembling DNA molecules that may be configured into a wide variety of shapes and may be used to deliver molecules to cells (Zhang, et al. ACS Nano 8(7): 6633-43, 2014). A skilled artisan will be able to determine with no more than routine experimentation an appropriate type or types of nanoparticle with which to deliver a composition of the invention.

Some embodiments of the invention include use of a vector for delivery of nucleic acid sequences encoding a fusion protein of the invention into cells. Vectors suitable for use in methods of the invention are known and routinely used in the art, including expression vectors, etc. A non-limiting example of a vector delivery means that may be used in certain embodiments of methods of the invention, are viral vectors. Numerous adenovirus-based delivery systems are routinely used in the art for deliver to, for example, lung, liver, the central nervous system, endothelial cells, and muscle. Non-limiting examples of viral vectors that may be used in compositions and methods of the invention are: adeno-associated virus (AAV) vectors; a helper-dependent or gutless adenovirus; pox virus vectors such as an orthopox, e.g., vaccinia virus vectors, a Modified vaccinia Ankara (MVA), NYVAC, or avipox, e.g. canary pox or fowl pox; polyoma virus vectors; papilloma virus vectors; picornavirus vectors; herpes simplex virus (HSV) vectors; and SV 40 vectors. Viral vectors may include regulatory elements, such as promoters, enhancers, etc., which may be selected to provide constitutive or regulated/inducible expression. Viral vector systems, and the use of promoters and enhancers, etc. are routine in the art and can be used in conjunction with methods and compositions described herein.

In certain embodiments of the invention, a vector is used to deliver a nucleic acid sequence encoding a fusion protein of the invention into a cell. Compositions of the invention, in some embodiments, include a vector comprising a nucleic acid sequence encoding such a fusion protein. In some embodiments, the nucleic acid sequence is operatively linked to a promoter sequence. Preparation and use of such vectors for delivering sequences into a cell and or subject are well known in the art. Vectors can be used in methods of the invention that result in transient expression of an mRNA, for example, for at least 1, 2, 3, 4, 5, 6, 7, 8, 9, 10 or more hours, or for at least 1, 2, 3, 4, 5, 6, 7, 8, 9, 10 or more weeks. The length of the transient expression can be determined using routine methods based on elements such as, but not limited to the specific vector construct selected and the target cell and/or tissue.

Vector delivery may be systemic, such as by intravenous or intramuscular administration, or by any other means that allows for introduction into a desired target cell. Some embodiments of the invention include methods of delivering the nucleic acid molecule comprising a fusion protein into cells using a vector and such vectors may be in a pharmaceutically acceptable carrier that may, but need not, include a slow release matrix in which the gene delivery vehicle is imbedded. In some embodiments, a vector can be produced from a recombinant cell, and a pharmaceutical composition of the invention may include one or more cells that produced the vector. In some embodiments, a vector may be encapsulated in a nanoparticle, including but not limited to an inhalable nanoparticle. One of skill in the art will be able to determine with no more than routine experimentation an appropriate type or types of vector, nanoparticle, and/or other means suitable for delivering a composition of the invention to a cell and/or subject.

In some embodiments of methods of the invention, a composition of the invention is delivered into a cell using a physical delivery method such as, but not limited to: heat shock, electroporation, biolistics, microinjection, sonoporation, photoporation, magnetofection, needle injection, jet injection, electrofection, hydrofection, and optofection. Non-limiting art-known methods of physical delivery methods that may be used in certain embodiments of methods of the invention are set forth in Herrero M. J., et al. (2017) Physical Methods of Gene Delivery. In: Brunetti-Pierri N. (eds) Safety and Efficacy of Gene-Based Therapeutics for Inherited Disorders. Springer, Cham. pp 113-135, the content of which is incorporated herein by reference in its entirety. Non-limiting examples of cells to which a composition of the invention can be physically delivered are provided elsewhere herein.

### Cells and Subjects

It will be understood that a cell included in a composition or method of the invention may be one of a plurality of cells. As used herein, the term “plurality” means two or more. For example, though not intended to be limiting, a plurality may be at least 2, 3, 4, 5, 6, 7, 8, 9, 10, 15, 20, 25, 30, 35, 40, 45, 50, 100, 150, 250, 500, 1000, 10,000, 20,000, or 50,000, including each integer in this range. In some embodiments of the invention, a plurality of cells is all of the same cell type and all are infected with virus. In other embodiments of the invention, a plurality of cells comprises a mixed plurality of cells, meaning not all cells need to be the same cell type. A cell used in an embodiment of a composition or method of the invention may be one or more of: a single cell, an isolated cell, a cell that is one of a plurality of cells, a cell that is one of two or more cells that are in physical contact with each other, etc.

In some embodiments, a cell is a cell in an organism. In certain embodiments, delivering a composition of the invention into an organism comprises delivering the composition into a cell in the organism. In a non-limiting example, delivery of a composition of the invention in to a cell in an organism comprises a physical delivery method, non-limiting examples of which are set forth elsewhere herein. In some embodiments, a cell into which a composition of the invention is delivered is a germline cell, a stem cell, an oocyte, or an embryonic cell.

In some embodiments of the invention, a composition of the invention is delivered into a cell to produce a transgenic cell, and/or into an organism to produce a transgenic organism. As used herein, the term “transgenic” means that one or more DNA sequences have been introduced to a cell or organism by artificial means. In a non-limiting example, an organism may be made transgenic by having a small sequence of exogenous DNA injected into a fertilized egg or developing embryo. As used herein the term “exogenous” means not naturally present in a cell and/or organism. For example, an exogenous polynucleotide sequence can be a polynucleotide sequence that is not naturally present in the cell and/or organism, respectively. Another non-limiting example of an exogenous molecule is a protein expressed in a cell and/or organism that is not naturally expressed in that cell or organism. In an embodiment of a transgenic organism, when the organism can transmit that extra piece of DNA to its offspring and/or descendants. A transgenic plant may be prepared using a method of the invention by introducing exogenous DNA into one or more different cells and/or tissues of a plant. Information regarding preparing transgenic organisms is known in the art, for example, see Amare Bihon Asfaw & Ayalew Assefa; Pedro González-Redondo (Reviewing editor) (2019) Animal transgenesis technology: A review, Cogent Food & Agriculture, 5:1, the content of which is incorporated by reference herein in its entirety.

In some aspects of the invention, a cell is obtained from a living subject or is an isolated cell. An isolated cell may be a primary cell, such as those recently isolated from an animal (e.g., cells that have undergone none or only a few population doublings and/or passages following isolation), or may be a cell of a cell line that is capable of prolonged proliferation in culture (e.g., for longer than 3 months) or indefinite proliferation in culture (immortalized cells). In some embodiments of the invention, a cell is somatic. Somatic cells may be obtained from an individual, e.g., a subject and cultured according to standard cell culture protocols known to those of ordinary skill in the art. A cell or plurality of cells may be obtained from a surgical specimen, tissue, or cell biopsy, etc.

In some embodiments of the invention, a cell is a healthy normal cell, which is not known to have one or more of a viral infection, disease, disorder, or abnormal condition. In some embodiments, a cell comprising a composition of the invention or used in conjunction with a method of the invention comprises an abnormal cell, for example, a cell comprising a viral infection, a cell obtained from a subject diagnosed as having or suspected of having a viral infection. In some embodiments of the invention, a cell is a control cell, a non-limiting example of which is a cell known not to be a virally infected cell.

A cell used in an embodiment of a method of the invention may comprise one or a plurality of a human cell. Non-limiting examples of a cell that may be used in a composition or embodiment of a method of the invention are one or more of a eukaryotic cell, a vertebrate cell, which in some embodiments of the invention is a mammalian cell. Non-limiting examples of a cell that may comprise an embodiment of a composition of the invention or be used in a method of the invention are a vertebrate cell, an invertebrate cell, a non-human primate cell, a rodent cell, dog cell, cat cell, avian cell, fish cell, a cell obtained from a wild animal, a cell obtained from a domesticated animal, or another suitable cell of interest. In some embodiments of the invention, a cell is a lung cell, an alveolar epithelial cell, a type I pneumocyte, a type II pneumocyte, a club cell, an alveolar macrophage, a cuboidal ciliated epithelial cell, or an endothelial cell, or a cardiomyocyte. In some embodiments of the invention, a cell is a neuronal cell, a glial cell, or other type of central nervous system (CNS) or peripheral nervous system (PNS) cell. In some embodiments of the invention, a cell is an embryonic stem cell or embryonic stem cell-like cell. In some embodiments of the invention, a cell is a natural cell and in certain embodiments of the invention, a cell is an engineered cell.

Cells comprising embodiments of compositions of the invention or used in methods of the invention may be obtained from normal or diseased tissue, and may be maintained in cell culture following their isolation. In certain embodiments of the invention, a cell is a free cell in culture, a free cell obtained from a subject, a cell obtained in a solid biopsy from a subject, organ, or solid culture, etc. A cell comprising an embodiment of the invention or used in a method of the invention may be genetically modified or not genetically modified.

As used herein, the term “subject” may refer to human or non-human animals, including mammals and non-mammals, vertebrates and invertebrates, and may also refer to any multicellular organism or single-celled organism such as a eukaryotic (including plants and algae) or prokaryotic organism, archaeon, microorganisms (e.g., bacteria, archaea, fungi, protists, viruses), and aquatic plankton. Non-limiting examples of mammalian subjects include primates (including but not limited to humans and non-human primates), rodents (including but not limited to mice, rats, squirrels, chipmunks, prairie dogs), bats, lagomorphs, deer, canids, felids, bears, horses, cows, sheep, goats, and pigs. A subject may be considered to be a normal subject or may be a subject known to have or suspected of having a disorder, disease, or condition. Non-limiting examples of diseases or conditions include infectious diseases, such as SARS-CoV, SARS-CoV-2, and human immunodeficiency virus (HIV).

### Controls

Certain embodiments of compositions and methods of the invention used to suppress a viral infection comprise comparing results obtained for a cell or plurality of cells, or a subject, with a control value obtained from a control cell or plurality of control cells, or a control subject. As a non-limiting example, some embodiments of the invention include contacting a plurality of cells with either a composition of the invention comprising a nanoparticle and an mRNA encoding an N-Nuc fusion protein or with a nanoparticle that did not contain said mRNA, infecting all cells with SARS-CoV-2, and determining the respective proportions of infected cells. A control may also be a no-treatment control, wherein a cell or subject does not receive a treatment.

As used herein a control may be as described above and, in addition, may be a predetermined value, which can take a variety of forms. It can be a single cut-off value, such as a median or mean, or a reference value or range of values. In some embodiments of the invention, a control value is a value determined previously for a subject during the course of infection and/or treatment of the subject. A control can be established based upon comparative groups such as a cell (in vitro or in a subject) that is contacted with a composition of the invention, compared to a cell or subject that under substantially identical conditions is not contacted with, or is contacted with a different amount of the composition of the invention. Another example of comparative groups may include a cell or subject that has a disorder or condition and a cell or subject without the disorder or condition. Another comparative group may be a subject or cells from a subject with a family history of a disease or condition and a subject or cells from a subject without such a family history. Other examples of comparative groups may include, but are not limited to cells or subjects that have a severe viral infection; cells or subjects that do not have a severe viral infection; cells or subjects that are asymptomatic for a viral infection, etc. Those in the art will readily identify suitable control cells and subjects for use with compositions and methods of the invention. In some embodiments, a control may be a placebo, an inactive substance used to compare results with a composition or method of the invention.

### EXAMPLES

### Example 1. Assays for In Vitro Function

### Materials and Methods

Generation of Nanoparticle-Capsuled mRNA

Non-His-tagged mRNAs encoding N-Nuc, N-EGFP, and N-Nuc mut polypeptides were synthesized by TriLink Biotechnologies (TriLink Biotechnologies, San Diego, CA), and were subsequently encapsulated into polymer-based nanoparticles (NPs), including poly(beta-amino ester) (PBAE) and lipid-based nanoparticles (LNPs). Hyperbranched PBAE (hDD90-118) polymer was synthesized following a method previously described (Patel, et al. Adv Mater. 1805116-1805123, 2019). Briefly, acrylate:backbone amine:trifunctional amine monomers were reacted at a ratio of 1:0.5:0.2. Monomers were stirred in anhydrous dimethylformamide at a concentration of 150 mg/mL at 40° C. for 4 hours (h) then 90° C. for 48 h. The mixtures were allowed to cool to 30° C. and end cap amine was added at 1.5 molar equivalent relative to the excess acrylate and stirred for a further 24 h. The polymers were purified by dropwise precipitation into cold anhydrous diethyl ether spiked with glacial acetic acid, vortexed, and centrifuged at 1250×g for 2 min to pellet the polymer. The supernatant was discarded and polymer washed twice more in fresh diethyl ether and dried under vacuum for 48 h. Polymers were stored at −20° C. PBAE nanoparticles were formulated at 0.5 mg/mL of mRNA with 50 to 1 PBAE polymer to mRNA by mass, in 0.1M of sodium acetate buffer (pH 5.2).

LNP formulations were prepared using a method previously described. Briefly, lipids were dissolved in ethanol at molar ratios of 35:16:46.5:2.5 (ionizable:helper:cholesterol:PEG) and were nanoprecipitated by mixing with mRNA in acetate buffer (pH 5.0) in a ratio of 3:1 (aqueous:ethanol). Formulations were dialyzed against PBS (pH 7.4) in dialysis cassettes for at least 18 h. Formulations were concentrated using Amicon Ultra Centrifugal filters (Millipore Sigma, Burlington, MA), passed through a 0.22-μm filter, and stored at 4° C. until use. All formulations were tested for particle size, RNA encapsulation, and endotoxin, and were found to be 80-120 nm in size, with >80% encapsulation and <10 endotoxin units/ml.

### Cell Culture and Transfection

Vero E6 cells were grown in Dulbecco's Modified Eagles Medium (DMEM; Gibco Fisher Scientific, Waltham, MA) supplemented with 10% fetal bovine serum (FBS; Gibco Fisher Scientific, Waltham, MA) and incubated in a humidified 5% CO₂ incubator. Cells were plated on 96-well plates for optimization of transfection conditions and virus therapeutic tests. mRNAs were delivered to cells using either a TransIT®-mRNA Transfection Kit (Minis Bio, Madison, WI) or polymer-based nanoparticles (NPs) including poly(beta-amino ester) (PBAE) or lipid-based nanoparticles (LNPs).

### Viral Inhibition Assay, Cell Staining and Imaging

Vero E6 cells were seeded in 96-well plates and 100 ng or 200 ng of mRNA was transfected by PBAEs or LNPs per well. Two separate transfection plates were prepared. One plate was transfected 24 h prior to infection, the other was transfected three hours prior to infection. Plates were moved into a BSL4 lab (National Emerging Infectious Diseases Laboratory (NEIDL), Boston University, Boston, MA), washed with PBS to remove residual mRNA in media, and infected with SARS-CoV-2 USA-WA1/2020 at MOIs of 0.2 and 0.04 (5× high and 1× low, respectively), along with uninfected controls. Fifteen hours later, the inoculum was removed, cells were washed in PBS to remove input virus, and fresh DMEM was added back. Twenty four hours after the media change, supernatant was collected from the wells, diluted 1:200 and 1:1000, and the viral supernatant dilutions were added to new, untransfected Vero E6 cells. The progeny plates were fixed in 10% formalin at 24 hours post infection (hpi). This process was repeated on the progenitor plates at 48 hours after the media change. Plates were removed from containment and were immunostained to detect SARS-CoV-2 infection. Cells were permeabilized in 0.1% Triton™ X-100 for 15 minutes, followed by blocking in 3.5% BSA for 1 hour. Progeny plates were stained with anti-SARS-CoV-2 N protein antibody at 1:10,000 (R004; Sino Biologicals, Beijing, China) overnight at 4° C. Cells were washed in PBS and incubated in an Alexa Fluor 546-conjugated anti-rabbit secondary antibody (ThermoFisher Scientific, Waltham, MA) for 2 hours at room temperature. After 3×5 min washes, Hoechst 33342 was added to stain cell nuclei. Plates were imaged on an automated imager (BioTek Cytation 1; BioTek, Winooski, VT). The images were put through a custom CellProfiler (Broad Institute, Cambridge, MA) pipeline to count the number of infected cells and total nuclei, and the ratio of infected cells/total cells was calculated and used as the readout to determine viral infectivity.

### Protein Purification

The genes encoding staphylococcus nuclease (Nuc), N-Nuc, Nuc mut (equivalent to Nuc with E43 S and R87G), and N-Nuc mut polypeptides were codon-optimized for expression in bacteria. The genes were then de novo synthesized by Epoch Life Science (Epoch Life Science, Missouri City, TX), and cloned into pBad-HisD vector (Invitrogen, Waltham, MA) with a 5′ His-tag gene. E. coli strain NEB 10-beta (New England Biolabs, Ipswich, MA) was transformed with the aforementioned plasmids and then cultured overnight on agar plates containing LB and ampicillin at 37° C. Single colonies were picked and grown overnight in 150 mL of LB medium with 400 μg/ml carbenicillin (Goldbio, St. Louis, MO) and 0.02% arabinose (Sigma-Aldrich, St. Louis, MO) at 37° C. followed by additional 24-hr culturing at room temperature. Bacteria were harvested at 5,000 rpm, 4° C. for 15 min, lysed using xTractor™ Buffer (Takara Bio USA, Mountain View, CA). Proteins were then purified using Capturem™ His-Tagged Purification Maxiprep Kit (Takara Bio USA, Mountain View, CA) following the manufacturer's instructions. N-Nuc and N-Nuc mut lysates were heated at 70° C. for 15 min to disrupt the self-assembling of N-protein before purification. The buffer of the purified protein was exchanged to 10 mM MOPs and 100 mM NaCl, pH 7.2 with centrifugal concentrators (GE Healthcare Life Sciences, Millipore Sigma, Burlington, MA). Purified proteins were confirmed by SDS-PAGE electrophoresis. Protein concentrations were quantified by BCA protein assay using a Pierce™ BCA Protein Assay Kit (ThermoFisher Scientific, Waltham, MA).

### Nuclease Activity Assay

For DNA cleavage assay, 600 ng pcDNA-3.1 plasmids were mixed with purified nucleases or N protein linked nucleases in the reaction solution (25 mM Tris-HCl, pH 8.0 with either 10 mM of CaCl₂ or EGTA). The solutions were then incubated at 37° C. for 20 min (or 40 min) followed by the addition of stop reaction (50 mM EDTA, final concentration). Nuclease activities were determined by the detection of the disappearance of DNA bands after running DNA electrophoresis with 1% agarose gel.

For RNA cleavage assay, an RNA reporter (5′-/5HEX/uuuuuuuu/3IABkFQ/-3′ [SEQ ID NO: 14]), synthesized by IDT (Integrated DNA Technologies, Coralville, IA) was mixed with each of the four purified proteins in the reaction solution (25 mM Tris-HCl, pH 8.0 with either 10 mM of CaCl₂ or EGTA) followed by a 5 min incubation at 37° C. Fluorescence of the reaction solutions was then measured by a Tecan Spark plate reader (excitation: 535 nm, emission: 556 nm; Tecan, Mannedorf, Switzerland) to determine nuclease activity.

### Statistical Analysis

All data are shown as mean±standard deviation (s.d.). Twelve replicates of each treatment condition were performed for antiviral activity assays.

### Results and Discussion

### Antiviral Activity

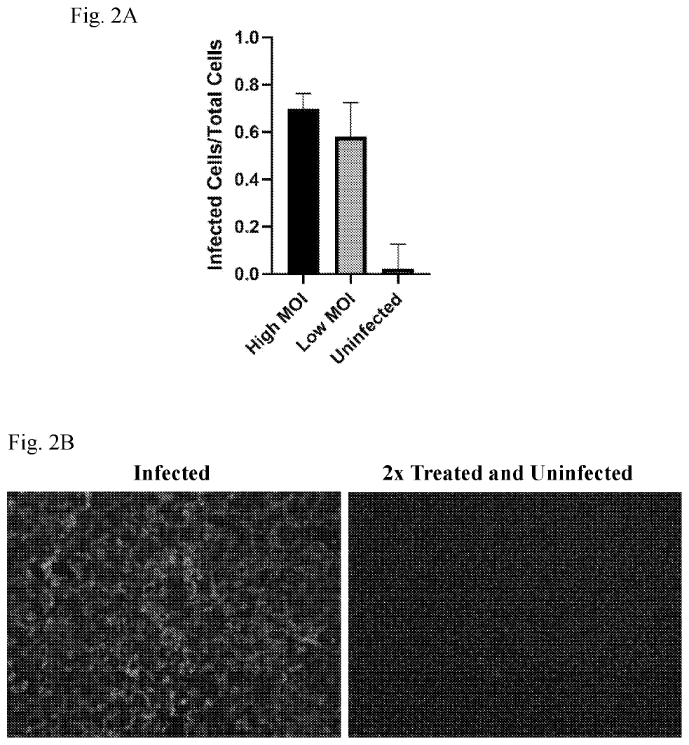

Experiments were performed to determine baseline results of SARS-CoV-2 infection at low (0.04, 1×) and high MOI (0.2, 5×). An average of ˜70% of cells were infected at high MOI versus an average of −58% at low MOI (FIG. 2A, values shown are means of 12 replicates for each treatment condition). Antibody staining of SARS-CoV-2 N-protein was also performed on untreated infected cells (Low MOI) and treated uninfected cells (2× 200 ng N-Nuc treated) showing no background signal from the antibody (FIG. 2B).

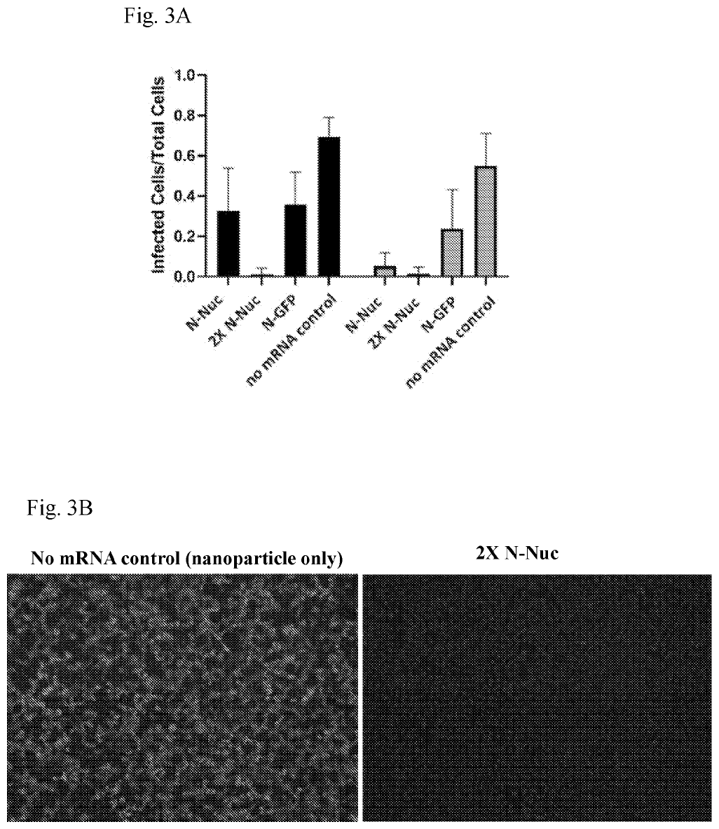

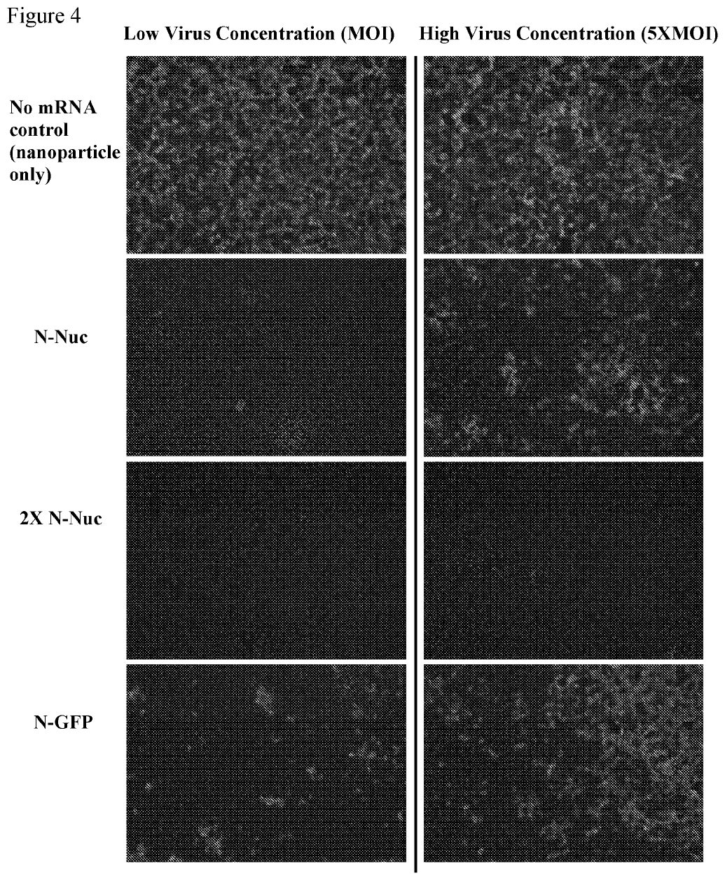

Experiments were then performed in which cells were transfected with hPBAE nanoparticles containing mRNA encoding the N-Nuc fusion protein and then infected with SARS-CoV-2 at either a high or low MOI (0.2 or 0.04, respectively) three hours after mRNA transfection (FIG. 3A-B). N-Nuc mRNA was delivered at either a 1× (100 ng) or 2× dose (200 ng). Cells were harvested 1 day post-infection (DPI), and counted or fixed and stained. At both high and low MOI, both the 1× and 2× doses of N-Nuc mRNA showed a reduced proportion of infected cells compared to the no-mRNA controls (FIG. 3A [values shown are means of 12 replicates for each treatment condition], FIG. 4). Cells transfected with N-Nuc mRNA showed minimal to no N-Nuc staining, compared to no-mRNA controls (FIG. 3B, FIG. 4). Results of transfection efficiency tests indicated that transfected cells were healthy.

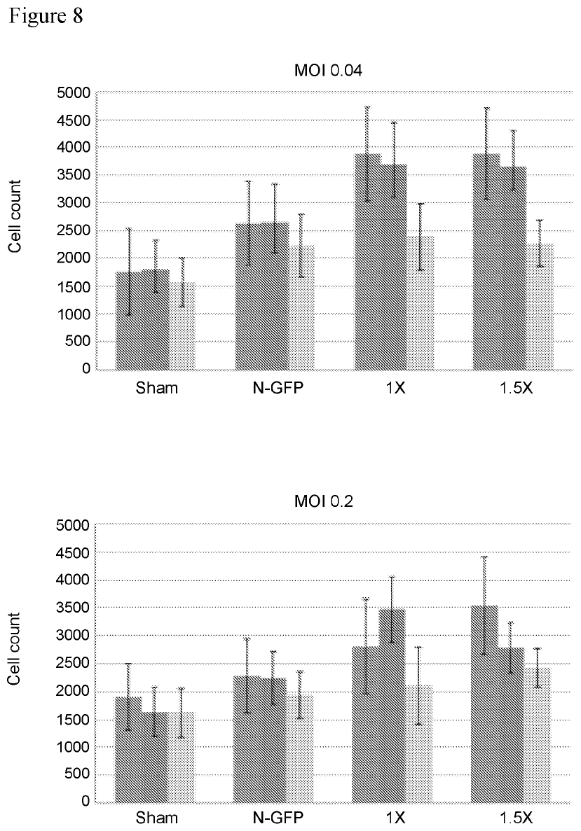

Additional transfection experiments were performed in which cells were transfected with either a 1× (200 ng) or a 1.5× (300 ng) dose of N-Nuc mRNA plus a standard mRNA transfection reagent (Minis TransIT®-mRNA Transfection Kit; Mirus Bio, Madison, WI) instead of with inhalable nanoparticles, and were subsequently infected with SARS-CoV-2 at either a low or high MOI (0.04 and 0.2, respectively) (FIG. 8). Cells were harvested at 0, 1, and 2 dpi. On average when treated with N-Nuc mRNA, cell death was reduced by ˜57% when infected at high MOI and an average of ˜63% when infected at low MOI.

### Calcium-Dependent Nuclease Activity Assays

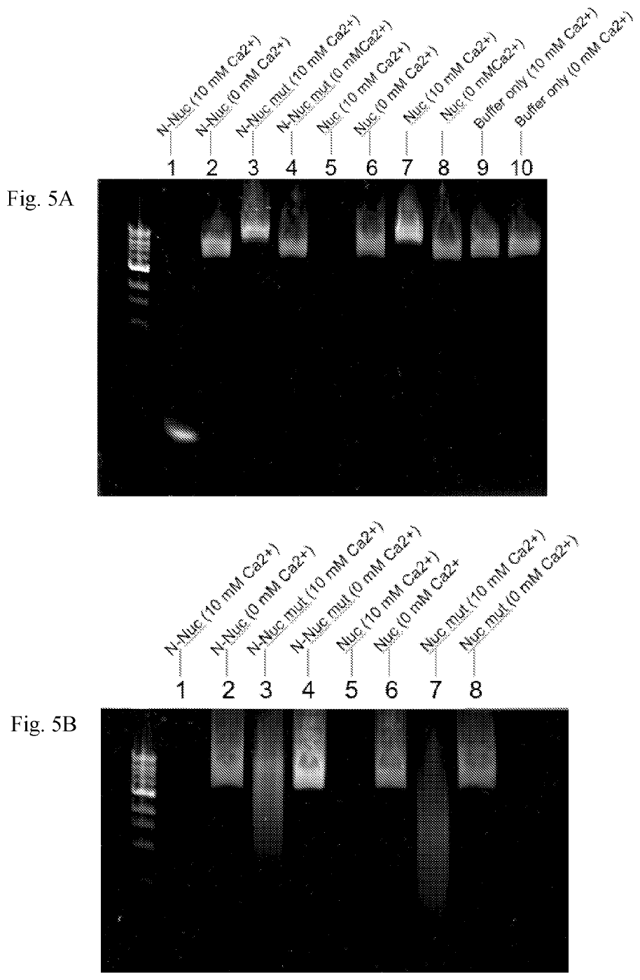

To evaluate the calcium-dependent nuclease activity of the N-Nuc fusion protein, its ability to cleave both DNA and RNA was tested and was compared to the calcium-dependent nuclease activity of wild-type SN (“Nuc”), an SN mutant with 10⁶ lower nuclease activity (“Nuc mut”; Weber et al., Biochemistry 30(25), 6103-6114, 1991), and an N-Nuc mut fusion protein. Calcium-dependent DNA cleavage activity was tested under two different sets of conditions, “low” and “high” (FIGS. 5A-B). Under the “low” set of conditions (FIG. 5A), 600 ng plasmid was incubated with either 200 ng N-Nuc fusion protein, 200 ng N-Nuc mut fusion protein, 100 ng Nuc, or 100 ng Nuc mut. A set of two reactions was performed for each protein, one in the presence of 10 mM Ca²⁺ and one in the presence of 0 mM Ca²⁺. All “low” reactions were incubated at 37° C. for 20 minutes. Under the “high” set of conditions (FIG. 5B), 600 ng plasmid was incubated with either 1000 ng N-Nuc fusion protein, 1000 ng N-Nuc mut fusion protein, 500 ng Nuc, or 500 ng Nuc mut. A set of two reactions was performed for each protein, one in the presence of 10 mM Ca²⁺ and one in the presence of 0 mM Ca²⁺. All “high” reactions were incubated at 37° C. for 40 minutes. Under both sets of conditions, the N-Nuc fusion protein successfully digested plasmid DNA in the presence of 10 mM Ca²⁺ (FIGS. 5A-B, lanes 1 and 5).

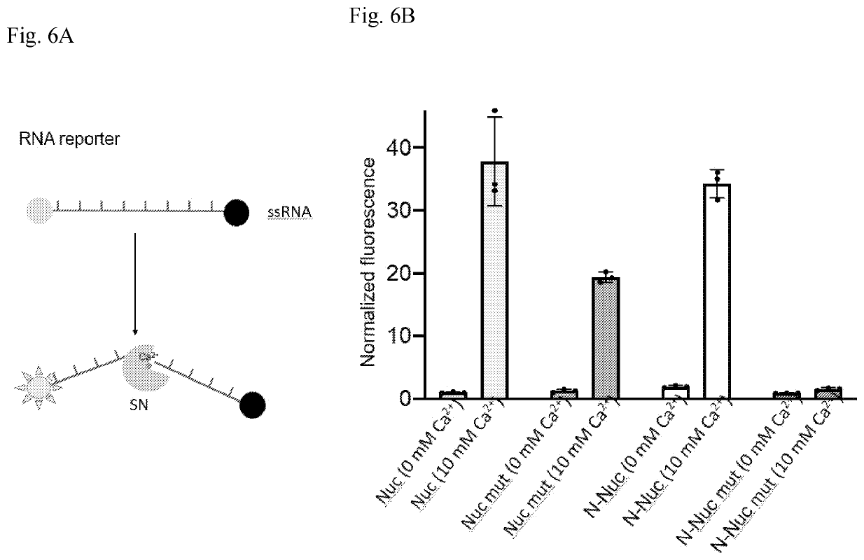

To test calcium-dependent RNA cleavage activity, a single-stranded (ssRNA) oligo reporter was used which had a HEX™ fluorophore on its 5′ end and an Iowa Black® FQ quencher (Integrated DNA Technologies, Coralville, IA) on its 3′ end. As illustrated in FIG. 6A, cleavage of the ssRNA oligo separated the fluorophore from the quencher, resulting in increased fluorescence over a baseline value. For each reaction, 250 nM ssRNA reporter was incubated with either 200 ng N-Nuc, 200 ng N-Nuc mut, 100 ng Nuc, or 100 ng Nuc mut in 20 μl-solution. A set of two reactions was performed for each protein, one in the presence of 10 mM Ca²⁺ and one in the presence of 0 mM Ca²⁺. All reactions were incubated at 37° C. for 5 minutes. As measured by normalized fluorescence, both the wild-type Nuc and N-Nuc fusion protein demonstrated comparable cleavage efficiency in the presence of 10 mM Ca²⁺ (FIG. 6B).

### Protein Purification

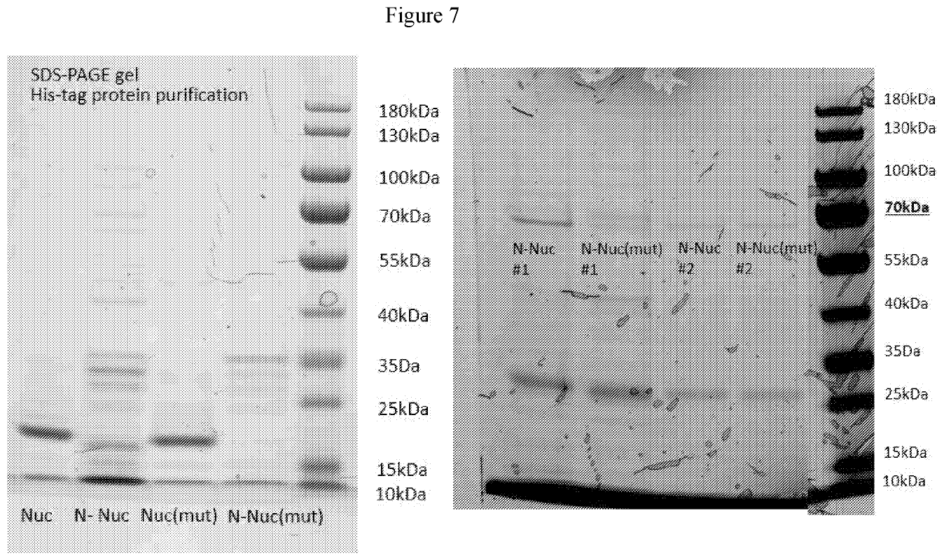

To investigate whether N-Nuc fusion proteins were capable of self-oligomerizing and/or aggregating, His-tagged wild-type Nuc, Nuc mut, N-Nuc, and N-Nuc mut proteins were expressed and either directly purified following expression (FIG. 7, left panel) or incubated at 70° C. for 15 minutes before purification (FIG. 7, right panel). Results of both experiments were run on SDS-PAGE gels. Incubated fusion proteins showed higher molecular weight bands (˜70 kDa) that were not observed in the non-incubated counterparts, suggesting that N-protein fusion proteins did self-oligomerize and/or aggregate.

### Example 2. In Vivo Experiments

### Materials and Methods

Materials and methods are as described in Example 1 herein.

### Animal Model

N-Nuc mRNA is introduced to K18-hACE2 transgenic mice (Jackson Laboratory, Bar Harbor, ME) by nebulization, followed by intranasal infection with 10⁵ FFUs of SARS-CoV-2. At four days post infection, mice are sacrificed and respiratory systems harvested. Respiratory tissue from each mouse is divided into two samples. The first sample is placed in PBS, ground up with a TissueLyser II (Qiagen, Germantown, MD), and an FFU assay is performed to determine the amount of infectious viral particles present. The second respiratory tissue sample is fully lysed and RNA is harvested. qPCR is performed to detect the number of SARS-CoV-2 genomes present in the tissue.

### Results

Fewer infectious viral particles are confirmed to be present in respiratory tissue from treated mice. Fewer SARS-CoV-2 genomes are confirmed to be present in respiratory tissue from treated mice.

### EQUIVALENTS

Although several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto; the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

All references, patents and patent applications and publications that are cited or referred to in this application are incorporated by reference in their entirety herein.

What is claimed is:

1. A composition for suppressing a viral infection, the composition comprising a nucleic acid molecule encoding a fusion protein, wherein the fusion protein comprises a protein capable of self-assembling/oligomerizing with a protein of a viral particle operatively linked to a molecule capable of inhibiting a function of the viral particle.

2. The composition of claim 1, wherein the viral infection is at least one of an RNA virus infection, a DNA virus infection, a coronavirus infection, a retroviral infection, and a SARS-CoV-2 infection.

3. The composition of claim 1, wherein the inhibited function of the viral particle comprises one or more of: reproduction of the viral particle within a cell, assembling a new viral particle, releasing a new viral particle from a cell, and packaging a viral genome.

4. The composition of claim 1, wherein the protein capable of self-assembling/oligomerizing with the viral particle protein is a viral nucleocapsid (N) protein, and optionally is a SARS-CoV-2 N protein.

5. The composition of claim 1, wherein the nucleic acid molecule comprises a sequence encoding one or more of SEQ ID NO: 4, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, and SEQ ID NO: 15.

6. The composition of claim 1, wherein the molecule capable of inhibiting the function of the viral particle is a protease, a peptide, an antibody, a lipase, or a nuclease.

7. The composition of claim 6, wherein the nuclease is an RNase, a DNase, or a micrococcal nuclease.

8. The composition of claim 6, wherein the nuclease is a calcium (Ca²⁺)-dependent nuclease.

9. The composition of claim 1, wherein the operatively linked viral particle protein is not a capsid protein.

10. A cell comprising the composition of claim 1.

11. A fusion protein encoded by the composition of claim 1.

12. A cell comprising the fusion protein of claim 11.

13. A virus particle comprising the fusion protein encoded by the composition of claim 1.

14. A method of treating a subject known to have, suspected of having, or at risk of having a viral infection, the method comprising administering to the subject a composition comprising:
(i) a nucleic acid molecule encoding a fusion protein, wherein the fusion protein comprises a protein capable of self-assembling/oligomerizing with a protein of a viral particle operatively linked to a molecule capable of inhibiting a function of the viral particle, and
(ii) a delivery molecule,
in an amount effective to treat the viral infection.

15. A method of treating a subject known to have, suspected of having, or at risk of having a viral infection, the method comprising administering to the subject a composition in an amount effective to treat the viral infection, wherein the composition comprises an exogenous nucleic acid molecule encoding a fusion protein comprising a protein capable of self-assembling/oligomerizing with a protein of a viral particle, wherein the protein is operatively linked to a molecule capable of inhibiting a function of the viral particle, and wherein a means of administering the composition comprises a physical delivery means.

16. The method of claim 14, wherein the viral infection is an RNA virus infection, a DNA virus infection, a coronavirus infection, or a retroviral infection, and optionally is a SARS-CoV-2 infection.

17. The method of claim 14, wherein the administered composition is a composition comprising a nucleic acid molecule encoding a fusion protein, wherein the fusion protein comprises a protein capable of self-assembling/oligomerizing with a protein of a viral particle operatively linked to a molecule capable of inhibiting a function of the viral particle.

18. The method of claim 14, wherein the administration delivers the composition into a cell of the subject, and optionally wherein the cell is germline cell, an embryonic cell, a stem cell, or a reproductive cell.

19. A method of inhibiting infectivity of a mutated first virus, the method comprising: expressing in a cell comprising a virus particle of the mutated first virus, a fusion protein comprising a polypeptide capable of self-assembling/oligomerizing with a protein of the first virus and operatively linked to a nuclease enzyme, wherein the mutated first virus comprises the protein of the first virus and the composition inhibits infectivity of the mutated first virus, wherein optionally the fusion protein is encoded by the nucleic acid of the composition of claim 1.

20. The method of claim 19, wherein the polypeptide capable of self-assembling/oligomerizing with a protein comprises a protein of the first virus.


SEQUENCE LISTING (15 sequences)

SEQ ID NO: 1 - protein, 419 aa, SARS-CoV-2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 2 - protein, 12 aa, Artificial Sequence
  Synthetic oligonucleotide
GGSGGTGSGGSG

SEQ ID NO: 3 - protein, 149 aa, Staphylococcus aureus
ATSTKKLHKEPATLIKAIDGDTVKLMYKGQPMTFRLLLVDTPETKHPKKGVEKYGPEASAFTKKMVENAKKIEVEFDKGQRTDKYGRGLAYIYADGKMVNEALVRQGLAKVAYVYKPNNTHEQHLRKSEAQAKKEKLNIWSEDNADSGQ

SEQ ID NO: 4 - protein, 580 aa, Artificial sequence
  Synthetic oligonucleotide
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAGGSGGTGSGGSGATSTKKLHKEPATLIKAIDGDTVKLMYKGQPMTFRLLLVDTPETKHPKKGVEKYGPEASAFTKKMVENAKKIEVEFDKGQRTDKYGRGLAYIYADGKMVNEALVRQGLAKVAYVYKPNNTHEQHLRKSEAQAKKEKLNIWSEDNADSGQ

SEQ ID NO: 5 - DNA, 1743 bp, Artificial sequence
  Synthetic oligonucleotide
atgtccgacaacggcccgcagaatcaacgaaacgctccccgaattacattcggtggtccatcagactccactggaagtaatcagaatggggaacggtcaggcgccagatcaaagcaacggcgaccgcagggtctcccgaataataccgcttcctggtttaccgccctcacccagcatggcaaagaagacctgaagtttccaaggggtcagggggtacccattaatactaattcctccccagacgaccagattggctactatcggcgagcgaccaggcgcatacggggtggtgatgggaaaatgaaggatctttcacctagatggtatttttactatctgggtacgggtccggaagcgggtctgccctacggcgccaacaaggacggtataatatgggttgcaactgaaggagctttgaatacacctaaggaccacattggcactcgaaatcccgccaacaatgcagccatcgttttgcagctgccgcagggtactacacttccaaagggtttctatgccgagggatctagaggcgggtcacaggcatcctcccggtcaagttcaagatctcgaaattcatctcgcaacagtaccccaggctcttctagaggtacctcaccggcccggatggccggcaacggtggcgatgctgctctcgcgctcctcctcctggaccgccttaatcagctggaaagtaagatgtccgggaagggacaacaacaacaaggccagacagtaactaaaaagagcgcggcagaagcgtcaaagaagccccgacaaaaaaggactgctacaaaggcatataatgtcacccaggctttcggccgcagaggcccagagcagacccagggcaactttggtgatcaagagttgatccggcagggcactgactataaacactggccccagatcgcccaattcgcaccaagtgcgagcgcatttttcgggatgtctaggattggaatggaagtcactccatctggtacctggttgacatacacaggtgcgataaagctggacgacaaggatccgaactttaaagatcaggttattttgctcaacaaacacatcgatgcttacaagactttcccccctactgaacccaaaaaggataaaaagaagaaagcggacgaaacacaggcgttgccgcagagacagaaaaaacagcaaactgtgactcttcttccggcggcagacttggatgatttctctaagcaactgcagcagagtatgtcctctgctgactcaacgcaagccggcgggagtggtggtacgggaagcgggggcagcggcgctacatcaacaaagaaactgcacaaggaaccagctactcttatcaaagctattgacggcgatactgtgaaacttatgtacaaagggcaaccaatgacatttcggcttttgctggtagatactccagaaacaaagcatcctaagaagggggtagagaaatacggcccggaggcgtctgcatttacaaaaaaaatggtcgaaaatgctaagaaaattgaagtcgagttcgataaaggtcagcggacggacaagtatggaaggggattggcttatatatatgccgacggaaaaatggtaaatgaagcattggtgcgccaggggttggcaaaagtggcctacgtctacaaaccgaataacacccatgaacagcatttgagaaaatctgaagcccaggcgaagaaagagaaactcaacatctggtcagaagataatgctgacagtggacaataa

SEQ ID NO: 6 - protein, 127 aa, SARS-CoV-2
TASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEG

SEQ ID NO: 7 - protein, 119 aa, SARS-CoV-2
TKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPP

SEQ ID NO: 8 - protein, 55 aa, SARS-CoV-2
PTEPKKDKKKKADETQALPQRQKKQQTVTLLPAADLDDFSKQLQQSMSSADSTQA

SEQ ID NO: 9 - protein, 49 aa, SARS-CoV-2
MSDNGPQNQRNAPRITFGGPSDSTGSNQNGERSGARSKQRRPQGLPNNT

SEQ ID NO: 10 - protein, 73 aa, SARS-CoV-2
GSRGGSQASSRSSSRSRNSSRNSTPGSSRGTSPARMAGNGGDAALALLLLDRLNQLESKMSGKGQQQQGQTVT

SEQ ID NO: 11 - protein, 28 aa, SARS-CoV-2
PTEPKKDKKKKADETQALPQRQKKQQTV

SEQ ID NO: 12 - protein, 28 aa, SARS-CoV-2
VTLLPAADLDDFSKQLQQSMSSADSTQA

SEQ ID NO: 13 - protein, 149 aa, Staphylococcus aureus
ATSTKKLHKEPATLIKAIDGDTVKLMYKGQPMTFRLLLVDTPSTKHPKKGVEKYGPEASAFTKKMVENAKKIEVEFDKGQRTDKYGGGLAYIYADGKMVNEALVRQGLAKVAYVYKPNNTHEQHLRKSEAQAKKEKLNIWSEDNADSGQ

SEQ ID NO: 14 - RNA, 8 nt, Artificial sequence
  Synthetic oligonucleotide
uuuuuuuu

SEQ ID NO: 15 - protein, 173 aa, SARS-CoV-2
TKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA